(19) United States
(12) Patent Application Publication
(10) Pub. No.: US 2023/0345986 A1
(43) Pub. Date: Nov. 2, 2023

(54) CONJUGATED DIYNES AND THEIR USE AS FLAVOR MODIFIERS

(71) Applicant: Firmenich SA, Satigny (CH)

(72) Inventors: Wen-Juan XIANG, Shanghai (CN); Dan-Ting YIN, Shanghai (CN)

(73) Assignee: Firmenich SA, Satigny (CH)

(21) Appl. No.: 18/014,789

(22) PCT Filed: Oct. 26, 2021
(86) PCT No.: PCT/EP2021/079668

§ 371 Date: Jan. 6, 2023

(30) Foreign Application Priority Data
Oct. 27, 2020 (CN) CN2020/123890
Nov. 11, 2020 (EP) 20206828.4

(51) Int. Cl. A23L 27/00; C07C 225/16; C07D 317/50; C07D 207/04; C07D 207/325; C07D 207/18; C07D 211/36; C07D 211/72

(52) U.S. Cl. CPC A23L 27/88; C07C 225/16; C07D 317/50; C07D 207/04; C07D 207/325; C07D 207/18; C07D 211/36; C07D 211/72

(57) ABSTRACT

The present disclosure generally provides conjugates diynes, particularly 6,8-diyne amides, and the use of such compounds and related compounds as flavor modifiers. In some aspects, the disclosure provides compositions that include such conjugated diynes, such as compositions that include such conjugated diynes and one or more additional compounds, such as a sweetener, salt, a glutamate, an arginate, and the like. In some other aspects, the disclosure provides methods of reducing or eliminating the amount of sweetener, salt, glutamate, or arginate in a food or beverage product.

### CROSS-REFERENCE TO RELATED APPLICATIONS

The present application claims the benefit of priority of PCT Application No. PCT/CN2020/123890, filed Oct. 27, 2020, and European Patent Application No. 20206828.4, filed Nov. 11, 2020, both of which are hereby incorporated by reference as though set forth herein in their entireties.

### TECHNICAL FIELD

The present disclosure generally provides conjugates diynes, particularly 6,8-diyne amides, and the use of such compounds and related compounds as flavor modifiers. In some aspects, the disclosure provides compositions that include such conjugated diynes, such as compositions that include such conjugated diynes and one or more additional compounds, such as a sweetener, salt, a glutamate, an arginate, and the like. In some other aspects, the disclosure provides methods of reducing or eliminating the amount of sweetener, salt, glutamate, or arginate in a food or beverage product.

### DESCRIPTION OF RELATED ART

The taste system provides sensory information about the chemical composition of the external world. Taste transduction is one of the more sophisticated forms of chemically triggered sensation in animals. Signaling of taste is found throughout the animal kingdom, from simple metazoans to the most complex of vertebrates. Mammals are believed to have five basic taste modalities: sweet, bitter, sour, salty, and umami/kokumi.

Umami is the taste most commonly associated with the savory taste of monosodium glutamate (MSG), meat products, cheeses, tomatoes, mushrooms, soy sauce, fish sauce, miso, and the like. Mammals generally perceive umami to be a pleasurable sensation. Kokumi is a related taste commonly associated with the taste of fermented products, soy sauce, fish sauce, and shrimp paste. Many of these typical sources of umami and kokumi taste are high in glutamic acid and salt, or rely on animal products.

Excessive sodium intake can cause a number of health-related problems. One such problem is hypertension. Hypertension is a condition in which the pressure of the blood against artery walls is high enough that it may eventually cause heart disease and other health problems. Excessive sodium intake also adversely affect the balance of water and minerals in the body. For example, excessive sodium intake can cause calcium loss, which can lead to osteoporosis and other problems. Excessive consumption of food products containing glutamic acid can also have certain adverse health effects, as glutamic acid interferes with the functioning of neurotransmitters. Thus, it is generally desirable to reduce the consumption of sodium and glutamic acid. Further, there is increased consumer demand for food and beverage products that do not contain animal-derived ingredients, such as animal-derived fats, meat products, or dairy products.

Enhancement of salt, umami, or kokumi flavors provides an alternative approach to partially or completely replacing ingredients that are traditionally used to impart salt, umami, and/or kokumi taste. Even so, there is a limited number of compounds, especially naturally derived compounds, that can accomplish this effectively. Thus, there is a continuing need to discover new compounds having utility as flavor modifiers, especially compounds that enhance, among other flavors, the taste of salt, umami, kokumi, or any combination thereof.

### SUMMARY

The present disclosure relates to the discovery that certain compounds exhibit a desirable and surprising effect of enhancing umami and/or salty flavor when used in a food or beverage product.

In a first aspect, the disclosure provides compounds of formula (I):

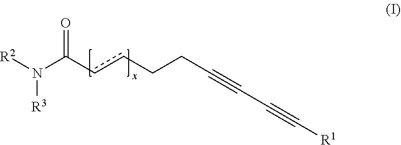

wherein:
R¹ is a hydrogen atom, C₁₋₆ alkyl, C₂₋₆ alkenyl, C₃₋₁₀ cycloalkyl, C₂₋₁₀ heterocyclyl, C₆₋₁₄ aryl, C₁₋₁₂ heteroaryl, or —X¹—R⁴, wherein the alkyl and alkenyl groups are each optionally substituted one or more times by substituents selected independently from R^{X}, and wherein the cycloalkyl, heterocyclyl, aryl, and heteroaryl groups are each optionally substituted one or more times by substituents selected independently from the group consisting of R^{Y};
R² is a hydrogen atom, C₁₋₆ alkyl, or C₂₋₆ alkenyl, wherein the alkyl and alkenyl groups are optionally substituted one or more times by substituents selected independently from R^{X};
R³ is a hydrogen atom, C₁₋₆ alkyl, C₂₋₆ alkenyl, C₃₋₁₀ cycloalkyl, C₂₋₁₀ heterocyclyl, C₆₋₁₄ aryl, C₁₋₁₂ heteroaryl, or —X²—R⁵, wherein the alkyl and alkenyl groups are each optionally substituted one or more times by substituents selected independently from R^{X}, and wherein the cycloalkyl, heterocyclyl, aryl, and heteroaryl groups are each optionally substituted one or more times by substituents selected independently from the group consisting of R^{Y};
X¹ and X² are independently C₁₋₆ alkylene or C₂₋₆ alkenylene, each of which is optionally substituted one or more times by substituents selected independently from R^{X};
R⁴ and R⁵ are independently C₃₋₁₀ cycloalkyl, C₂₋₁₀ heterocyclyl, C₆₋₁₄ aryl, or C₁₋₁₂ heteroaryl, each of which is optionally substituted one or more times by substituents selected independently from R^{Y};
R^{X} is a halogen atom, oxo, —CN, nitro, —OH, —NH₂, —C(O)H, —O—C(O)H, —C(O)—OH, —NH—C(O)H, —C(O)—NH₂, —O—(C₁₋₆ alkyl), —NH—(C₁₋₆ alkyl), —N(C₁₋₆ alkyl)₂, —C(O)—(C₁₋₆ alkyl), —O—C(O)—(C₁₋₆ alkyl), —NH—C(O)—(C₁₋₆ alkyl), —C(O)—O—(C₁₋₆ alkyl), —C(O)—NH—(C₁₋₆ alkyl), —C(O)—N(C₁₋₆ alkyl)₂, —S(O)₂—(C₁₋₆ alkyl), —O—S(O)₂—(C₁₋₆ alkyl), —NH—S(O)₂—(C₁₋₆ alkyl), —S(O)₂—O—(C₁₋₆ alkyl), —S(O)₂—NH—(C₁₋₆ alkyl), —S(O)₂—N(C₁₋₆ alkyl)₂, C₃₋₁₀ cycloalkyl, C₂₋₁₄ heterocyclyl, C₆₋₁₄ aryl, C₂₋₁₄ heteroaryl, C₁₋₆ alkyl, C₂₋₆ alkenyl, C₁₋₆ haloalkyl, C₂₋₆ haloalkenyl, C₁₋₆ haloalkoxy, C₂₋₆ haloalkenyloxy, or (C₁₋₆ alkoxy)-C₁₋₆ alkyl;
R^{Y} is a halogen atom, oxo, —CN, nitro, —OH, —NH₂, —C(O)H, —O—C(O)H, —C(O)—OH, —NH—C(O)H, —C(O)—NH₂, —O—(C₁₋₆ alkyl), —NH—(C₁₋₆ alkyl), —N(C₁₋₆ alkyl)₂, —C(O)—(C₁₋₆ alkyl), —O—C(O)—(C₁₋₆ alkyl), —NH—C(O)—(C₁₋₆ alkyl), —C(O)—O—(C₁₋₆ alkyl), —C(O)—NH—(C₁₋₆ alkyl), —C(O)—N(C₁₋₆ alkyl)₂, —S(O)₂—(C₁₋₆ alkyl), —O—S(O)₂—(C₁₋₆ alkyl), —NH—S(O)₂—(C₁₋₆ alkyl), —S(O)₂—O—(C₁₋₆ alkyl), —S(O)₂—NH—(C₁₋₆ alkyl), —S(O)₂—N(C₁₋₆ alkyl)₂, C₃₋₁₀ cycloalkyl, C₂₋₁₄ heterocyclyl, C₆₋₁₄ aryl, C₂₋₁₄ heteroaryl, C₁₋₆ alkyl, C₂₋₆ alkenyl, C₁₋₆ haloalkyl, C₂₋₆ haloalkenyl, C₁₋₆ haloalkoxy, C₂₋₆ haloalkenyloxy, or (C₁₋₆ alkoxy)-C₁₋₆ alkyl; and
x is 1 or 2;
wherein R² and R³ optionally combine to form a heterocyclic or heteroaromatic ring comprising at least one nitrogen atom and from 2 to 10 carbon atoms, and wherein the ring is optionally substituted one or more times by substituents selected independently from R^{Y}; and
wherein the dotted-line bond indicates an optional carbon-carbon double bond, which can exist in either the E or Z configuration.

In a second aspect, the disclosure provides uses of any compounds of the first aspect, and any embodiments thereof.

In a third aspect, the disclosure provides uses of any compounds of the first aspect, and any embodiments thereof, to modify a flavor of an ingestible composition. In some embodiments, the ingestible composition is a flavored product such as a flavored food or beverage product.

In a fourth aspect, the disclosure provides uses of any compounds of the first aspect, and any embodiments thereof, to enhance a salty taste of an ingestible composition. In related aspects, the disclosure provides uses of any compounds of the first or second aspects, and any embodiments thereof, to reduce the salt (e.g., sodium chloride) content of an ingestible composition. In some embodiments of these aspects, the ingestible composition comprises sodium chloride. In some embodiments, the ingestible composition is a flavored product such as a flavored food or beverage product.

In a fifth aspect, the disclosure provides uses of any compounds of the first aspect, and any embodiments thereof, to enhance an umami taste of an ingestible composition. In related aspects, the disclosure provides uses of any compounds of the first or second aspects, and any embodiments thereof, to reduce or eliminate the glutamate or aspartate content of an ingestible composition. In some embodiments of these aspects, the ingestible composition is substantially free of monosodium glutamate (MSG). In some embodiments, the ingestible composition is a flavored product such as a flavored food or beverage product.

In a sixth aspect, the disclosure provides uses of any compounds of the first aspect, and any embodiments thereof, to enhance a warming or heating effect of an ingestible composition. In some embodiments, the ingestible composition is a flavored product such as a flavored food or beverage product.

In a seventh aspect, the disclosure provides uses of any compounds of the first aspect, and any embodiments thereof, to enhance a cooling effect of an ingestible composition. In some embodiments of these aspects, the ingestible composition comprises sodium chloride. In some embodiments, the ingestible composition is a flavored product such as a flavored food or beverage product. In some embodiments, the ingestible composition is an oral care product, such as a mouthwash, a toothpaste, a whitening agent, a dentifrice, and the like. In some embodiments, the ingestible composition comprises menthol.

In an eighth aspect, the disclosure provides uses of any compounds of the first aspect to enhance a sweet taste of an ingestible composition. In related aspects, the disclosure provides uses of any compounds of the first or second aspects, and any embodiments thereof, to reduce or eliminate the sweetener (e.g., sucrose, fructose, sucralose, etc.) content of an ingestible composition. In some embodiments of these aspects, the ingestible composition is substantially free of caloric sweetener. In some embodiments, the ingestible composition is a flavored product such as a flavored food or beverage product.

In a ninth aspect, the disclosure provides uses of any compounds of the first aspect to reduce the sourness of an ingestible composition.

In a tenth aspect, the disclosure provides uses of any compounds of the first aspect to reduce the bitterness of an ingestible composition.

In an eleventh aspect, the disclosure provides methods of modifying the flavor of an ingestible composition, comprising introducing any compounds of the first aspect to an ingestible composition. In some embodiments, the ingestible composition is a food or beverage product.

In a twelfth aspect, the disclosure provides methods of enhancing a salty taste of an ingestible composition, comprising introducing any compounds of the first aspect to the ingestible composition. In a related aspect, the disclosure provides methods of reducing salt (e.g., sodium chloride) content of an ingestible composition, the method comprising introducing any compounds of the first aspect to the ingestible composition. In some embodiments, the ingestible composition is a food or beverage product.

In a thirteenth aspect, the disclosure provides methods of enhancing an umami taste of an ingestible composition, comprising introducing any compounds of the first aspect to the ingestible composition. In a related aspect, the disclosure provides methods of reducing or eliminating glutamate (e.g., monosodium glutamate) content of an ingestible composition, the method comprising introducing any compounds of the first aspect to the ingestible composition. In some embodiments, the ingestible composition is a food or beverage product.

In a fourteenth aspect, the disclosure provides methods of enhancing a kokumi taste of an ingestible composition, comprising introducing any compounds of the first aspect to the ingestible composition. In a related aspect, the disclosure provides methods of reducing or eliminating glutamyl (e.g., L-glutamyl peptides) content of an ingestible composition, the method comprising introducing any compounds of the first aspect to the ingestible composition. In another related aspect, the disclosure provides methods of reducing or eliminating animal (e.g., animal broth or meat) content of an ingestible composition, the method comprising introducing any compounds of the first aspect to the ingestible composition. In some embodiments, the ingestible composition is a food or beverage product.

In a fifteenth aspect, the disclosure provides methods of enhancing a warming or heating effect of an ingestible composition, comprising introducing any compounds of the first aspect to the ingestible composition. In some embodiments, the ingestible composition is a food or beverage product.

In a sixteenth aspect, the disclosure provides methods of enhancing a cooling effect of an ingestible composition, comprising introducing any compounds of the first aspect to the ingestible composition. In some embodiments, the ingestible composition is a food or beverage product. In some embodiments, the ingestible composition is an oral care product, such as a mouthwash, a toothpaste, a whitening agent, a dentifrice, and the like. In some embodiments, the ingestible composition comprises menthol.

In a seventeenth aspect, the disclosure provides methods of enhancing a sweet taste of an ingestible composition, comprising introducing any compounds of the first aspect to the ingestible composition. In a related aspect, the disclosure provides methods of reducing or eliminating sweetener (e.g., sucrose, fructose, sucralose, etc.) content of an ingestible composition, the method comprising introducing any compounds of the first aspect to the ingestible composition. In some embodiments, the ingestible composition is a food or beverage product.

In an eighteenth aspect, the disclosure provides methods of reducing a sour taste of an ingestible composition, comprising introducing any compounds of the first aspect to the ingestible composition. In some embodiments, the ingestible composition is a food or beverage product.

In a nineteenth aspect, the disclosure provides methods of reducing a bitter taste of an ingestible composition, comprising introducing any compounds of the first aspect to the ingestible composition. In some embodiments, the ingestible composition is a food or beverage product.

In a twentieth aspect, the disclosure provides compositions comprising any compounds of the first or second aspects. In some embodiments, the compounds of the first aspect make up at least 0.1% by weight, or at least 0.5% by weight, or at least 1.0% by weight, of the compositions on a dry weight basis (e.g., based on the total weight of the composition excluding the weight of any liquid carrier).

In a twenty-first aspect, the disclosure provides solid-state compositions comprising any compounds of the first or second aspects, wherein the compounds of the first aspect make up at least 0.1% by weight, or at least 0.5% by weight, or at least 1.0% by weight, of the solid-state compositions, based on the total weight of composition.

In a twenty-second aspect, the disclosure provides ingestible compositions comprising any compounds of the first or second aspects, wherein the concentration of the compounds of the first aspect in the ingestible compositions is no more than 200 ppm. In some embodiments, the ingestible composition is not a naturally occurring composition.

In a twenty-third aspect, the disclosure provides ingestible compositions comprising any compounds of the first aspect and a sweetener. In some embodiments, the sweetener is a caloric sweetener, such as sucrose, fructose, glucose, xylitol, erythritol, or combinations thereof. In some embodiments, the sweetener is a non-caloric sweetener, such as a steviol glycoside, a mogroside, aspartame, sucralose, acesulfame K, saccharin, or any combinations thereof. In some embodiments, the ingestible composition comprises one or more high-intensity sweeteners.

In a twenty-fourth aspect, the disclosure provides a concentrated flavoring composition comprising any compounds of the first aspect and a sweetener.

In a twenty-fifth aspect, the disclosure provides flavored products comprising any compositions of the preceding five aspects. In some embodiments, the flavored products are beverage products, such as soda, flavored water, tea, broth, and the like. In some other embodiments, the flavored products are food products, such as yogurt, soup, and the like.

Further aspects, and embodiments thereof, are set forth below in the Detailed Description, the Drawings, the Abstract, and the Claims.

### BRIEF DESCRIPTION OF THE DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the claimed inventions.

FIG. 1 shows chemical formulas representing compounds disclosed herein, wherein: R¹ is a hydrogen atom or an organic group; R² is a hydrogen atom, optionally substituted alkyl, or optionally substituted alkenyl; R³ is a hydrogen atom, or an organic group, where R² and R³ optionally combine to form an optionally substituted heterocyclic or heteroaromatic ring containing a nitrogen atom.

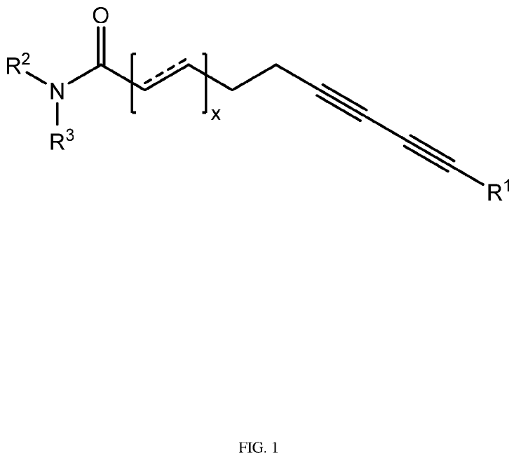

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary

As used herein, “solvate” means a compound formed by the interaction of one or more solvent molecules and one or more compounds described herein. In some embodiments, the solvates are ingestibly acceptable solvates, such as hydrates.

As used herein, “Cₐ to C_{b}” or “C_{a-b}” in which “a” and “b” are integers, refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

As used herein, “halogen” or “halo” means any one of the radio-stable atoms of column 7 of the Periodic Table of the Elements, such as fluorine, chlorine, bromine, or iodine. In some embodiments, “halogen” or “halo” refer to fluorine or chlorine.

As used herein, “alkyl” means a straight or branched hydrocarbon chain that is fully saturated (i.e., contains no double or triple bonds). In some embodiments, an alkyl group has 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like. Unless indicated to the contrary, the term “alkyl” refers to a group that is not further substituted.

As used herein, “substituted alkyl” means an alkyl group substituted with one or more substituents independently selected from C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, aryloxy (optionally substituted with halo, C₁-C₆ alkyl, C₁-C₆ alkoxy, C₁-C₆ haloalkyl, and C₁-C₆ haloalkoxy), C₃-C₇ carbocyclyloxy (optionally substituted with halo, C₁-C₆ alkyl, C₁-C₆ alkoxy, C₁-C₆ haloalkyl, and C₁-C₆ haloalkoxy), 3-10 membered heterocyclyl-oxy (optionally substituted with halo, C₁-C₆ alkyl, C₁-C₆ alkoxy, C₁-C₆ haloalkyl, and C₁-C₆ haloalkoxy), 5-10 membered heteroaryl-oxy (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkoxy (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkoxy (optionally substituted with halo, C₁-C₆ alkyl, C₁-C₆ alkoxy, C₁-C₆ haloalkyl, and C₁-C₆ haloalkoxy), aryl(C₁-C₆)alkoxy (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkoxy (optionally substituted with halo, C₁-C₆ alkyl, C₁-C₆ alkoxy, C₁-C₆ haloalkyl, and C₁-C₆ haloalkoxy), sulfhydryl (mercapto), halo(C₁-C₆)alkyl (e.g., —CF₃), halo(C₁-C₆)alkoxy (e.g., —OCF₃), C₁-C₆ alkylthio, arylthio (optionally substituted with halo, C₁-C₆ alkyl, C₁-C₆ alkoxy, C₁-C₆ haloalkyl, and C₁-C₆ haloalkoxy), C₃-C₇ carbocyclylthio (optionally substituted with halo, C₁-C₆ alkyl, C₁-C₆ alkoxy, C₁-C₆ haloalkyl, and C₁-C₆ haloalkoxy), 3-10 membered heterocyclyl-thio (optionally substituted with halo, C₁-C₆ alkyl, C₁-C₆ alkoxy, C₁-C₆ haloalkyl, and C₁-C₆ haloalkoxy), 5-10 membered heteroaryl-thio (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkylthio (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkylthio (optionally substituted with halo, C₁-C₆ alkyl, C₁-C₆ alkoxy, C₁-C₆ haloalkyl, and C₁-C₆ haloalkoxy), aryl(C₁-C₆)alkylthio (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkylthio (optionally substituted with halo, C₁-C₆ alkyl, C₁-C₆ alkoxy, C₁-C₆ haloalkyl, and C₁-C₆ haloalkoxy), amino, nitro, 0-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O).

As used herein, “alkoxy” means a moiety of the formula —OR wherein R is an alkyl, as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkylthio” means a moiety of the formula —SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” means a straight or branched hydrocarbon chain containing one or more double bonds. In some embodiments, the alkenyl group has from 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2-dienyl, and buta-1,2-dien-4-yl. Typical alkenyl groups include, but are in no way limited to, ethenyl, propenyl, butenyl, pentenyl, and hexenyl, and the like. Unless indicated to the contrary, the term “alkenyl” refers to a group that is not further substituted.

As used herein, “alkynyl” means a straight or branched hydrocarbon chain containing one or more triple bonds. In some embodiments, the alkynyl group has from 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂_₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like. Unless indicated to the contrary, the term “alkynyl” refers to a group that is not further substituted.

As used herein, “heteroalkyl” means a straight or branched hydrocarbon chain containing one or more heteroatoms, that is, an element other than carbon, including but not limited to, nitrogen, oxygen, and sulfur, in the chain backbone. In some embodiments, the heteroalkyl group has from 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain. Unless indicated to the contrary, the term “heteroalkyl” refers to a group that is not further substituted.

As used herein, “alkylene” means a branched or straight chain fully saturated di-radical chemical group containing only carbon and hydrogen that is attached to the rest of the molecule via two points of attachment (i.e., an alkanediyl). In some embodiments, the alkylene group has from 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene. Unless indicated to the contrary, the term “alkylene” refers to a group that is not further substituted.

As used herein, “alkenylene” means a straight or branched chain di-radical chemical group containing only carbon and hydrogen and containing at least one carbon-carbon double bond that is attached to the rest of the molecule via two points of attachment. In some embodiments, the alkenylene group has from 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl. Unless indicated to the contrary, the term “alkenylene” refers to a group that is not further substituted.

As used herein, “aromatic” means a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” means an aromatic ring or ring system (i.e., two or more fused rings that share two adjacent carbon atoms) containing only carbon in the ring backbone. When the aryl is a ring system, every ring in the system is aromatic. In some embodiments, the aryl group has from 6 to 18 carbon atoms, although the present definition also covers the occurrence of the term “aryl” where no numerical range is designated. In some embodiments, the aryl group has from 6 to 10 carbon atoms. The aryl group may be designated as “C₆₋₁₀ aryl,” “C₆-C₁₀ aryl,” or similar designations. Examples of aryl groups include, but are not limited to, phenyl, naphthyl, azulenyl, and anthracenyl. In some embodiments, the term “aryl” refers to phenyl. Unless indicated to the contrary, the term “aryl” refers to a group that is not further substituted

As used herein, “aryloxy” and “arylthio” mean moieties of the formulas RO— and RS—, respectively, in which R is an aryl as is defined above, such as “C₆₋₁₀ aryloxy” or “C₆₋₁₀ arylthio” and the like, including but not limited to phenyloxy and phenylthio.

As used herein “aralkyl” or “arylalkyl” means an aryl group connected, as a substituent, via an alkylene group, such as “C₇₋₁₄ aralkyl” and the like, including, but not limited to, benzyl, 2-phenylethyl, 3-phenylpropyl, and the like. In some embodiments, the alkylene group is a lower alkylene group (i.e., a C₁₋₄ alkylene group).

As used herein, “heteroaryl” means an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. In some embodiments, the heteroaryl group has from 5 to 18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has from 5 to 10 ring members or from 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl. Unless indicated to the contrary, the term “hereoaryl” refers to a group that is not further substituted.

As used herein, “heteroaralkyl” or “heteroarylalkyl” means heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. In some embodiments, the carbocyclyl group has from 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl. Unless indicated to the contrary, the term “carbocyclyl” refers to a group that is not further substituted

As used herein, “(carbocyclyl)alkyl” means a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a saturated or non-aromatic unsaturated carbocyclyl ring or ring system, according to any of the embodiments set forth above for carbocyclyl. Examples include cyclopropyl, cyclobutyl, cyclopentyl, and cyclohexyl. In some embodiments, the “cycloalkyl” groups are fully saturated. In some other embodiments, the “cycloalkyl” groups contain non-aromatic unsaturation. Such unsaturated cycloalkyl groups can alternately be referred to as “cycloalkenyl” groups.

As used herein, “cycloalkenyl” means a carbocyclyl ring or ring system having at least one double bond, wherein no ring in the ring system is aromatic, and according to any of the embodiments set forth above for carbocyclyl. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. In some embodiments, the heterocyclyl group has from 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “(heterocyclyl)alkyl” means a heterocyclyl group connected, as a substituent, via an alkylene group. Examples include, but are not limited to, imidazolinylmethyl and indolinylethyl.

An “acyl” group refers to a —C(═O)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-carbamyl” group refers to an “—N(R_{A})C(═O)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-thiocarbamyl” group refers to an “—N(R_{A})C(═S)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocycyl (optionally substituted with halo, C₁-C₆ alkyl, C₁-C₆ alkoxy, C₁-C₆ haloalkyl, and C₁-C₆ haloalkoxy), 3-10 membered heterocycyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy (optionally substituted with halo, C₁-C₆ alkyl, C₁-C₆ alkoxy, C₁-C₆ haloalkyl, and C₁-C₆ haloalkoxy), C₃-C₇ carbocyclyloxy (optionally substituted with halo, C₁-C₆ alkyl, C₁-C₆ alkoxy, C₁-C₆ haloalkyl, and C₁-C₆ haloalkoxy), 3-10 membered heterocyclyl-oxy (optionally substituted with halo, C₁-C₆ alkyl, C₁-C₆ alkoxy, C₁-C₆ haloalkyl, and C₁-C₆ haloalkoxy), 5-10 membered heteroaryl-oxy (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkoxy (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkoxy (optionally substituted with halo, C₁-C₆ alkyl, C₁-C₆ alkoxy, C₁-C₆ haloalkyl, and C₁-C₆ haloalkoxy), aryl(C₁-C₆)alkoxy (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkoxy (optionally substituted with halo, C₁-C₆ alkyl, C₁-C₆ alkoxy, C₁-C₆ haloalkyl, and C₁-C₆ haloalkoxy), sulfhydryl (mercapto), halo(C₁-C₆)alkyl (e.g., —CF₃), halo(C₁-C₆)alkoxy (e.g., —OCF₃), C₁-C₆ alkylthio, arylthio (optionally substituted with halo, C₁-C₆ alkyl, C₁-C₆ alkoxy, C₁-C₆ haloalkyl, and C₁-C₆ haloalkoxy), C₃-C₇ carbocyclylthio (optionally substituted with halo, C₁-C₆ alkyl, C₁-C₆ alkoxy, C₁-C₆ haloalkyl, and C₁-C₆ haloalkoxy), 3-10 membered heterocyclyl-thio (optionally substituted with halo, C₁-C₆ alkyl, C₁-C₆ alkoxy, C₁-C₆ haloalkyl, and C₁-C₆ haloalkoxy), 5-10 membered heteroaryl-thio (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkylthio (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkylthio (optionally substituted with halo, C₁-C₆ alkyl, C₁-C₆ alkoxy, C₁-C₆ haloalkyl, and C₁-C₆ haloalkoxy), aryl(C₁-C₆)alkylthio (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkylthio (optionally substituted with halo, C₁-C₆ alkyl, C₁-C₆ alkoxy, C₁-C₆ haloalkyl, and C₁-C₆ haloalkoxy), amino, amino(C₁-C₆)alkyl, nitro, 0-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

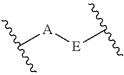

includes the substituent being oriented such that the A is attached at the leftmost attachment point of the molecule as well as the case in which A is attached at the rightmost attachment point of the molecule.

As used herein, a squiggly bond adjacent to a carbon-carbon double bond indicates that the substituents around the carbon-carbon double bond may connect in either an E or Z configuration, or a combination of molecules in either configuration.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, certain substituents or linking groups having only a single atom may be referred to by the name of the atom. For example, in some cases, the substituent “—H” may be referred to as “hydrogen” or “a hydrogen atom,” the substituent “—F” may be referred to as “fluorine” or “a fluorine atom,” and the linking group “—O—” may be referred to as “oxygen” or “an oxygen atom.”

Points of attachment for groups are generally indicated by a terminal dash (—) or by an asterisk (*). For example, a group such as *—CH₂—CH₃ or —CH₂—CH₃ both represent an ethyl group.

Chemical structures are often shown using the “skeletal” format, such that carbon atoms are not explicitly shown, and hydrogen atoms attached to carbon atoms are omitted entirely. For example, the structure

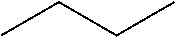

represents butane (i.e., n-butane). Furthermore, aromatic groups, such as benzene, are represented by showing one of the contributing resonance structures. For example, the structure

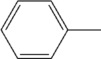

represents toluene.

As used herein, the term “conjugated diyne” refers to compounds of formula (I), any salts thereof, or any generic or specific embodiments thereof.

Other terms are defined in other portions of this description, even though not included in this subsection.

### Conjugated Diynes

In a at least one aspect, the disclosure provides compounds of formula (I):

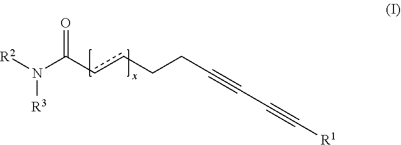

wherein:
R¹ is a hydrogen atom, C₁₋₆ alkyl, C₂₋₆ alkenyl, C₃₋₁₀ cycloalkyl, C₂₋₁₀ heterocyclyl, C₆₋₁₄ aryl, C₁₋₁₂ heteroaryl, or —X¹—R⁴, wherein the alkyl and alkenyl groups are each optionally substituted one or more times by substituents selected independently from R^{X}, and wherein the cycloalkyl, heterocyclyl, aryl, and heteroaryl groups are each optionally substituted one or more times by substituents selected independently from the group consisting of R^{Y};
R² is a hydrogen atom, C₁₋₆ alkyl, or C₂₋₆ alkenyl, wherein the alkyl and alkenyl groups are optionally substituted one or more times by substituents selected independently from R^{X};
R³ is a hydrogen atom, C₁₋₆ alkyl, C₂₋₆ alkenyl, C₃₋₁₀ cycloalkyl, C₂₋₁₀ heterocyclyl, C₆₋₁₄ aryl, C₁₋₁₂ heteroaryl, or —X²—R⁵, wherein the alkyl and alkenyl groups are each optionally substituted one or more times by substituents selected independently from R^{X}, and wherein the cycloalkyl, heterocyclyl, aryl, and heteroaryl groups are each optionally substituted one or more times by substituents selected independently from the group consisting of R^{Y};
X¹ and X² are independently C₁₋₆ alkylene or C₂₋₆ alkenylene, each of which is optionally substituted one or more times by substituents selected independently from R^{X};
R⁴ and R⁵ are independently C₃₋₁₀ cycloalkyl, C₂₋₁₀ heterocyclyl, C₆₋₁₄ aryl, or C₁₋₁₂ heteroaryl, each of which is optionally substituted one or more times by substituents selected independently from R^{Y};
R^{X} is a halogen atom, oxo, —CN, nitro, —OH, —NH₂, —C(O)H, —O—C(O)H, —C(O)—OH, —NH—C(O)H, —C(O)—NH₂, —O—(C₁₋₆ alkyl), —NH—(C₁₋₆ alkyl), —N(C₁₋₆ alkyl)₂, —C(O)—(C₁₋₆ alkyl), —O—C(O)—(C₁₋₆ alkyl), —NH—C(O)—(C₁₋₆ alkyl), —C(O)—O—(C₁₋₆ alkyl), —C(O)—NH—(C₁₋₆ alkyl), —C(O)—N(C₁₋₆ alkyl)₂, —S(O)₂—(C₁₋₆ alkyl), —O—S(O)₂—(C₁₋₆ alkyl), —NH—S(O)₂—(C₁₋₆ alkyl), —S(O)₂—O—(C₁₋₆ alkyl), —S(O)₂—NH—(C₁₋₆ alkyl), —S(O)₂—N(C₁₋₆ alkyl)₂, C₃₋₁₀ cycloalkyl, C₂₋₁₄ heterocyclyl, C₆₋₁₄ aryl, C₂₋₁₄ heteroaryl, C₁₋₆ alkyl, C₂₋₆ alkenyl, C₁₋₆ haloalkyl, C₂₋₆ haloalkenyl, C₁₋₆ haloalkoxy, C₂₋₆ haloalkenyloxy, or (C₁₋₆ alkoxy)-C₁₋₆ alkyl;
R^{Y} is a halogen atom, oxo, —CN, nitro, —OH, —NH₂, —C(O)H, —O—C(O)H, —C(O)—OH, —NH—C(O)H, —C(O)—NH₂, —O—(C₁₋₆ alkyl), —NH—(C₁₋₆ alkyl), —N(C₁₋₆ alkyl)₂, —C(O)—(C₁₋₆ alkyl), —O—C(O)—(C₁₋₆ alkyl), —NH—C(O)—(C₁₋₆ alkyl), —C(O)—O—(C₁₋₆ alkyl), —C(O)—NH—(C₁₋₆ alkyl), —C(O)—N(C₁₋₆ alkyl)₂, —S(O)₂—(C₁₋₆ alkyl), —O—S(O)₂—(C₁₋₆ alkyl), —NH—S(O)₂—(C₁₋₆ alkyl), —S(O)₂—O—(C₁₋₆ alkyl), —S(O)₂—NH—(C₁₋₆ alkyl), —S(O)₂—N(C₁₋₆ alkyl)₂, C₃₋₁₀ cycloalkyl, C₂₋₁₄ heterocyclyl, C₆₋₁₄ aryl, C₂₋₁₄ heteroaryl, C₁₋₆ alkyl, C₂₋₆ alkenyl, C₁₋₆ haloalkyl, C₂₋₆ haloalkenyl, C₁₋₆ haloalkoxy, C₂₋₆ haloalkenyloxy, or (C₁₋₆ alkoxy)-C₁₋₆ alkyl, and wherein any two adjacent R^{Y} on a ring may optionally combine to form a carbocyclic or heterocyclic ring; and
x is 1 or 2;
wherein R² and R³ optionally combine to form a heterocyclic or heteroaromatic ring comprising at least one nitrogen atom and from 2 to 10 carbon atoms, and wherein the ring is optionally substituted one or more times by substituents selected independently from R^{Y}; and
wherein the dotted-line bond indicates an optional carbon-carbon double bond, which can exist in either the E or Z configuration.

Note that the term “conjugated diynes,” as used herein, refers to compounds of formula (I), as defined above, or any embodiments thereof as set forth below.

R¹ can have any suitable value, according to the definition set forth above. In some embodiments, R¹ is a hydrogen atom. In some other embodiments, R¹ is C₁₋₆ alkyl or C₂₋₆ alkenyl, each of which is optionally substituted one or more times by substituents selected independently from R^{X}. In some further embodiments, R¹ is C₁₋₆ alkyl, which is optionally substituted one or more times by —OH, —O—(C₁₋₆ alkyl), or any combination thereof.

R² can have any suitable value, according to the definition set forth above. In some embodiments of any of the preceding embodiments, R² is a hydrogen atom. In some other embodiments of any of the preceding embodiments, R² is C₁₋₆ alkyl or C₂₋₆ alkenyl, each of which is optionally substituted one or more times by substituents selected independently from R^{X}. In some further such embodiments, R² is C₁₋₆ alkyl, which is optionally substituted one or more times by —OH, —O—(C₁₋₆ alkyl), or any combination thereof. In some further such embodiments, R² is unsubstituted C₁₋₆ alkyl, such as methyl, ethyl, isopropyl, propyl, butyl, sec-butyl, isobutyl, tert-butyl, and the like. In some embodiments, R² is methyl.

R³ can have any suitable value, according to the definition set forth above. In some embodiments of any of the preceding embodiments, R² and R³ is not both a hydrogen atom. Thus, if R² is a hydrogen atom, R³ is not a hydrogen atom.

In some embodiments of any of the preceding embodiments, R³ is C₁₋₆ alkyl or C₂₋₆ alkenyl, each of which is optionally substituted one or more times by substituents selected independently from R^{X}. In some further such embodiments, R³ is C₁₋₆ alkyl, which is optionally substituted one or more times by —OH, —O—(C₁₋₆ alkyl), oxo, or any combination thereof.

In some further such embodiments, R³ is unsubstituted C₁₋₆ alkyl, such as methyl, ethyl, isopropyl, propyl, butyl, sec-butyl, isobutyl, tert-butyl, pentyl, 2-methylbutyl, neopentyl, and the like. In some further such embodiments, R³ is unsubstituted linear C₄₋₇ alkyl, such as butyl, pentyl, hexyl, or heptyl. In some other such embodiments, R³ is unsubstituted branched C₃₋₆ alkyl. In some further such embodiments, R³ is a C₃₋₆ alkyl group branched at the 2-position, such as isobutyl or 2-methylbutyl. In some other such embodiments, R³ is a C₃₋₆ alkyl group branched at the Ψ-position (penultimate position), such as isobutyl, 3-methylbutyl, or 4-methylpentyl.

In some other embodiments, R³ is C₁₋₆ alkyl, which is substituted one or two times by —OH, oxo, or any combination thereof. In some such embodiments, R³ is 2-hydroxyethyl. In some other such embodiments, R³ is 2-hydroxy-2-methylpropyl. In some other such embodiments, R³ is 1,3-dihydroxy-2-methylpropyl. In some other such embodiments, R³ is 2-oxopropyl (—CH₂—C(═O)—CH₃).

In some embodiments, R³ is —X²—R⁵. In some such embodiments, X² is C₁₋₆ alkylene or C₂₋₆ alkenylene, which are optionally substituted one or more times by —OH, —O—(C₁₋₆ alkyl), oxo, or any combination thereof. In some further such embodiments, X² is unsubstituted C₁₋₆ alkylene, such as methylene or ethylene. In some further such embodiments, X² is unsubstituted C₂₋₆ alkenylene, such as ethenylene (—CH═CH—), for example, in the E configuration. In some other embodiments, X² is C₁₋₆ alkylene, which is substituted one or more times by —OH, —O—(C₁₋₆ alkyl), or any combination thereof, such as —CH(OH)—CH₂—, —CH₂—CH(OH)—, —CH(OCH₃)—CH₂—, or —CH₂—CH(OCH₃)—.

In some embodiments of any of the foregoing embodiments, R⁵ is a phenyl group, which is optionally substituted from one to three times by —OH, —O—(C₁₋₆ alkyl), or any combination thereof, wherein any two adjacent substituents can optionally combine to form a fused 5- or 6-membered ring having two oxygen atoms, such as a methylenedioxy group or a ethylenedioxy group. In some embodiments of any of the foregoing embodiments, R⁵ is 4-hydroxy-3-methoxyphenyl. In some embodiments of any of the foregoing embodiments, R⁵ is 3-hydroxy-4-methoxyphenyl. In some embodiments of any of the foregoing embodiments, R⁵ is phenyl. In some embodiments of any of the foregoing embodiments, R⁵ is 4-hydroxyphenyl. In some embodiments of any of the foregoing embodiments, R⁵ is 3-methoxyphenyl. In some embodiments of any of the foregoing embodiments, R⁵ is 3,4-dimethoxyphenyl. In some embodiments of any of the foregoing embodiments, R⁵ is 3,4-benzodioxyphenyl.

In some embodiments of any of the foregoing embodiments, R² and R³ combine to form a heterocyclic or heteroaromatic ring comprising at least one nitrogen atom and from 2 to 10 carbon atoms, and wherein the ring is optionally substituted one or more times by substituents selected independently from R^{Y}. In some embodiments, R² and R³ combine to form a heterocyclic or heteroaromatic ring selected from the group consisting of: 1-piperidinyl, 1-piperazinyl, 4-morpholinyl, 3,4-dihydropyridin-1(2H)-yl, 1-pyrrolidinyl, 1-imidazolidinyl, 1-pyrrazolidinyl, and 3-oxazolidinyl. In some further embodiments, R² and R³ combine to form a 1-piperidinyl ring. In some other such embodiments, R² and R³ combine to form a 3,4-dihydropyridin-1(2H)-yl ring. In some other such embodiments, R² and R³ combine to form a 1-pyrrolidinyl ring. In some other such embodiments, R² and R³ combine to form a 1H-pyrrol-1-yl ring. In some other such embodiments, R² and R³ combine to form a 2,3-dihydropyrrol-1(1H)-yl ring.

In some embodiments, x is 1, and the optional carbon-carbon double bond is absent. In some other embodiments, x is 2, and the optional carbon-carbon double bond is present.

Where the compounds disclosed herein have at least one chiral center, they may exist as individual enantiomers and diastereomers or as mixtures of such isomers. In some embodiments in connection with the second aspect, the sweet-enhancing compound has substantial enantiomeric purity.

Separation of the individual isomers or selective synthesis of the individual isomers is accomplished by application of various methods which are well known to practitioners in the art. Unless otherwise indicated (e.g., where the stereochemistry of a chiral center is explicitly shown), all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

In some embodiments, the compounds disclosed herein are capable of forming acid and/or base salts by virtue of the presence of amino and/or carboxyl groups or groups similar thereto. Physiologically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Physiologically acceptable salts can be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li⁺, Na⁺, K⁺, Mg²⁺ and Ca²⁺ and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the salts are comestibly acceptable salts, which are salts suitable for inclusion in comestible food and/or beverage products.

Table 1 provides examples of conjugated diynes. In some embodiments, the conjugated diyne is Compound 101. In some embodiments, the conjugated diyne is Compound 102. In some embodiments, the conjugated diyne is Compound 103. In some embodiments, the conjugated diyne is Compound 104. In some embodiments, the conjugated diyne is Compound 105. In some embodiments, the conjugated diyne is Compound 106. In some embodiments, the conjugated diyne is Compound 107. In some embodiments, the conjugated diyne is Compound 108. In some embodiments, the conjugated diyne is Compound 109. In some embodiments, the conjugated diyne is Compound 110. In some embodiments, the conjugated diyne is Compound 111. In some embodiments, the conjugated diyne is Compound 112. In some embodiments, the conjugated diyne is Compound 113. In some embodiments, the conjugated diyne is Compound 114. In some embodiments, the conjugated diyne is Compound 115. In some embodiments, the conjugated diyne is Compound 116. In some embodiments, the conjugated diyne is Compound 117. In some embodiments, the conjugated diyne is Compound 118. In some embodiments, the conjugates diyne is Compound 119. In some embodiments, the conjugates diyne is Compound 120. In some embodiments, the conjugates diyne is Compound 121. In some embodiments, the conjugates diyne is Compound 122. In some embodiments, the conjugates diyne is Compound 123. In some embodiments, the conjugates diyne is Compound 124. In some embodiments, the conjugates diyne is Compound 125. In some embodiments, the conjugates diyne is Compound 126. In some embodiments, the conjugates diyne is Compound 127. In some embodiments, the conjugates diyne is Compound 128. In some embodiments, the conjugates diyne is Compound 129. In some embodiments, the conjugates diyne is Compound 130.

TABLE 1

| No. | Structure |
|---|---|
| 101 | 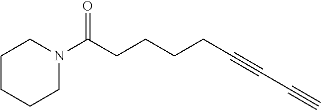 [SMILES: C#CC#CCCCCC(=O)N1CCCCC1] |
| 102 | 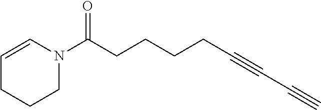 [SMILES: C#CC#CCCCCC(=O)N1C=CCCC1] |
| 103 | 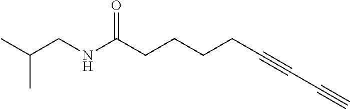 |
| 104 | 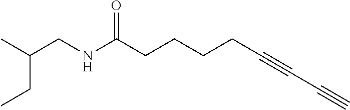 [SMILES: C#CC#CCCCCC(=O)NCC(C)CC] |
| 105 | 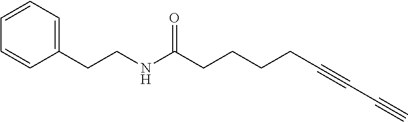 [SMILES: C#CC#CCCCCC(=O)NCCc1ccccc1] |
| 106 | 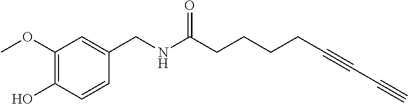 [SMILES: C#CC#CCCCCC(=O)NCc1ccc(O)c(OC)c1] |
| 107 | 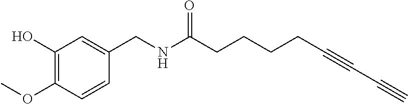 [SMILES: C#CC#CCCCCC(=O)NCc1ccc(OC)c(O)c1] |
| 108 | 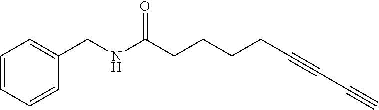 [SMILES: C#CC#CCCCCC(=O)NCc1ccccc1] |
| 109 | 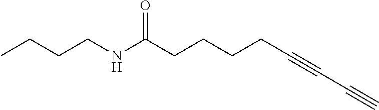 |
| 110 | 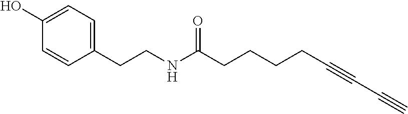 [SMILES: C#CC#CCCCCC(=O)NCCc1ccc(O)cc1] |
| 111 | 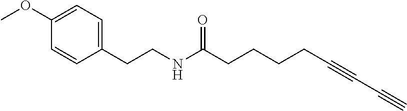 [SMILES: C#CC#CCCCCC(=O)NCCc1ccc(OC)cc1] |
| 112 | 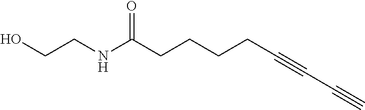 |
| 113 | 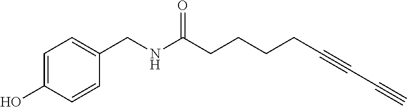 [SMILES: C#CC#CCCCCC(=O)NCc1ccc(O)cc1] |
| 114 | 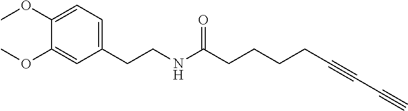 [SMILES: C#CC#CCCCCC(=O)NCCc1ccc(OC)c(OC)c1] |
| 115 | 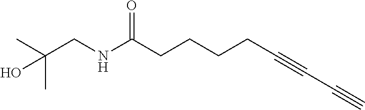 [SMILES: C#CC#CCCCCC(=O)NCC(C)(C)O] |
| 116 | 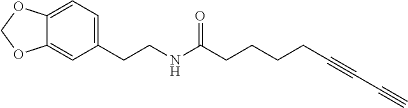 [SMILES: C#CC#CCCCCC(=O)NCCc1ccc2c(c1)OCO2] |
| 117 | 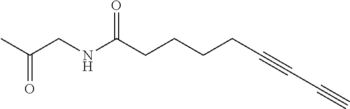 |
| 118 | 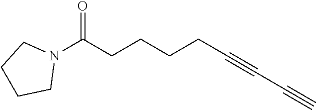 |
| 119 | 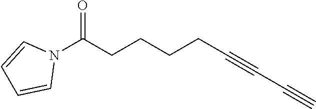 |
| 120 | 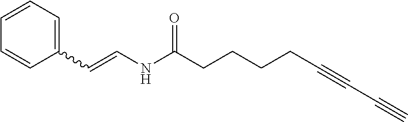 [SMILES: C#CC#CCCCCC(=O)NC=Cc1ccccc1] |
| 121 | 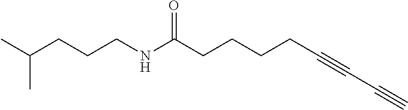 [SMILES: C#CC#CCCCCC(=O)NCCCC(C)C] |
| 122 | 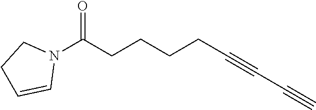 |
| 123 | 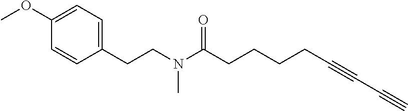 [SMILES: C#CC#CCCCCC(=O)N(C)CCc1ccc(OC)cc1] |
| 124 | 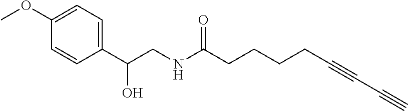 [SMILES: C#CC#CCCCCC(=O)NCC(O)c1ccc(OC)cc1] |
| 125 | 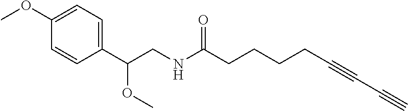 [SMILES: C#CC#CCCCCC(=O)NCC(OC)c1ccc(OC)cc1] |
| 126 | 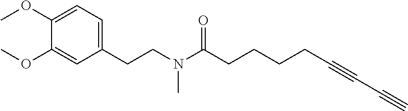 [SMILES: C#CC#CCCCCC(=O)N(C)CCc1ccc(OC)c(OC)c1] |
| 127 | 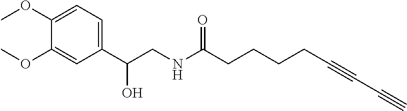 [SMILES: C#CC#CCCCCC(=O)NCC(O)c1ccc(OC)c(OC)c1] |
| 128 | 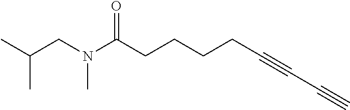 [SMILES: C#CC#CCCCCC(=O)N(C)CC(C)C] |
| 129 | 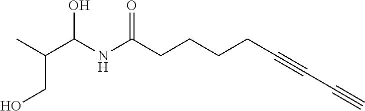 [SMILES: C#CC#CCCCCC(=O)NC(O)C(C)CO] |
| 130 | 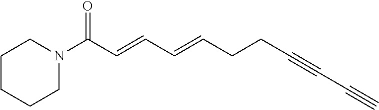 [SMILES: C#CC#CCC/C=C/C=C/C(=O)N1CCCCC1] |

### Solid State Forms and Solutions of Conjugated Diynes

In another aspect, the disclosure provides various solid-state forms of the conjugated diynes or their comestibly acceptable salts.

In some embodiments, the conjugated diynes or any of their comestibly acceptable salts exists as a crystalline solid, either in substantially pure form or in a formulation such as those set forth below. The crystalline solid can have any suitable polymorphic form, such as any polymorphic form obtainable via recrystallization in any suitable solvent system, according to techniques commonly used in the art of polymorph screening.

In some other embodiments, the conjugated diynes or any of their comestibly acceptable salts exists as an amorphous solid or a semi-amorphous solid, meaning that it lacks any regular crystalline structure. Such solids can be generated using standard techniques, such as spray drying, and the like.

In some embodiments, the conjugated diynes or any of their comestibly acceptable salts exists as a solvate, which is a pseudomorphic form of the compound in which one or more solvent molecules (such as water molecules) are taken up into the crystalline structure. Any suitable solvent or combination of solvents can be used, including, but not limited to, water, methanol, ethanol, n-propanol, isopropanol, n-butanol, 2-butanol, isobutanol, ethyl acetate, ethylene glycol, 1,2-propylene glycol, 1,3-propylene glycol, and the like. In some embodiments, the disclosure provides hydrates of the conjugates diyne or its comestibly acceptable salts. Such solvates can be generated by any suitable means, such as those techniques typically used by skilled artisans in the field of polymorph and solvate screening.

In some other embodiments, the conjugated diynes or any of their comestibly acceptable salts exist as a co-crystal with one or more other compounds, such as one or more other sweetener compounds. The conjugated diynes or any of their comestibly acceptable salts can form a co-crystal with any suitable compound. Non-limiting examples of such suitable compounds include fructose, glucose, galactose, sucrose, lactose, maltose, allulose, sugar alcohols (such as erythritol, sorbitol, xylitol, and the like), sucralose, steviol glycosides (such as rebaudioside A, rebaudioside E, rebaudioside M, and the like natural stevioside compounds), mogrosides (such as mogroside V, and other like natural mogroside compounds), aspartame, saccharin, acesulfame K, cyclamate, inulin, isomalt, and maltitol. Such co-crystals can be generated by any suitable means, such as those set forth in U.S. Patent Application Publication No. 2018/0363074, which is incorporated herein by reference.

In some embodiments, the conjugated diynes or their comestibly acceptable salts is in the form of a dry particle. Such dry particles can be formed by standard techniques in the art, such as dry granulation, wet granulation, and the like. Such particles can also contain a number of excipients, including, inert diluents, such as calcium carbonate, sodium carbonate, lactose, calcium phosphate, and sodium phosphate; granulating and disintegrating agents, such as starch, cellulosic materials, and alginic acid; binding agents, such as gelatin, guar gum, and acacia; and lubricating agents, such as magnesium stearate, stearic acid, and talc. Other excipients typically used in food and beverage products can also be included, such as typical foodstuff materials.

In some embodiments, the conjugated diynes or their comestibly acceptable salts are in the form of a liquid solution or a liquid suspension. Such compositions can also include: carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Such compositions can also include one or more coloring agents, one or more flavoring agents, and the like. Such liquid suspensions and solutions have a liquid carrier. In general, the liquid carrier comprises water. In some such cases, the liquid composition is an emulsion, such as an oil-in-water or a water-in-oil emulsion. Further, in some cases, water may be too polar to dissolve the conjugated diynes to the desired concentration. In such instances, it can be desirable to introduce water-miscible solvents, such as alcohols, glycols, polyols, and the like, to the solvent to enhance solubilization of the conjugated diynes.

In some embodiments, the conjugated diynes, or their comestibly acceptable salts, is in the form of a solution, i.e., are solvated within a liquid carrier. In some embodiments, the liquid carrier is an aqueous carrier. In some such embodiments, the solutions comprise a comestibly acceptable salt of an conjugated diyne, such as a hydrochloride salt, a potassium salt, or a sodium salt. Such solutions can be diluted to any suitable concentration.

### Uses, Methods, and Formulations

In other aspects, the disclosure provides formulations, uses, and methods of using the conjugated diynes or their comestibly acceptable salts (in any form according to the preceding aspects and embodiments thereof).

In certain aspects, the disclosure provides uses of any conjugated diynes of the preceding aspects or embodiments.

In certain aspects, the disclosure provides uses of any conjugated diynes of the preceding aspects or embodiments to modify a flavor of an ingestible composition. In some embodiments, the ingestible composition is a flavored product such as a flavored food or beverage product.

In certain aspects, the disclosure provides uses of any conjugated diynes of the preceding aspects or embodiments to enhance a salty taste of an ingestible composition. In related aspects, the disclosure provides uses of any conjugated diynes of the preceding aspects or embodiments and to reduce the salt (e.g., sodium chloride) content of an ingestible composition. In some embodiments of these aspects, the ingestible composition comprises sodium chloride. In some embodiments, the ingestible composition is a flavored product such as a flavored food or beverage product.

In certain aspects, the disclosure provides uses of any conjugated diynes of the preceding aspects or embodiments to enhance an umami taste of an ingestible composition. In related aspects, the disclosure provides uses of any conjugated diynes of the preceding aspects or embodiments to reduce or eliminate the glutamate or aspartate content of an ingestible composition. In some embodiments of these aspects, the ingestible composition is substantially free of monosodium glutamate (MSG). In some embodiments, the ingestible composition is a flavored product such as a flavored food or beverage product.

In certain aspects, the disclosure provides uses of any conjugated diynes of the preceding aspects or embodiments, to enhance a warming or heating effect of an ingestible composition. In some embodiments, the ingestible composition is a flavored product such as a flavored food or beverage product.

In certain aspects, the disclosure provides uses of any conjugated diynes of the preceding aspects or embodiments, to enhance a cooling effect of an ingestible composition. In some embodiments of these aspects, the ingestible composition comprises sodium chloride. In some embodiments, the ingestible composition is a flavored product such as a flavored food or beverage product. In some embodiments, the ingestible composition is an oral care product, such as a mouthwash, a toothpaste, a whitening agent, a dentifrice, and the like. In some embodiments, the ingestible composition comprises menthol.

In certain aspects, the disclosure provides uses of any compounds of the first or second aspects to enhance a sweet taste of an ingestible composition. In related aspects, the disclosure provides uses of any conjugated diynes of the preceding aspects or embodiments to reduce or eliminate the sweetener (e.g., sucrose, fructose, sucralose, etc.) content of an ingestible composition. In some embodiments of these aspects, the ingestible composition is substantially free of caloric sweetener. In some embodiments, the ingestible composition is a flavored product such as a flavored food or beverage product.

In certain aspects, the disclosure provides uses of any conjugated diynes of the preceding aspects or embodiments to reduce the sourness of an ingestible composition.

In certain aspects, the disclosure provides uses of any conjugated diynes of the preceding aspects or embodiments to reduce the bitterness of an ingestible composition.

In certain aspects, the disclosure provides methods of modifying the flavor of an ingestible composition, comprising introducing any conjugated diynes of the preceding aspects or embodiments to an ingestible composition. In some embodiments, the ingestible composition is a food or beverage product.

The disclosure also provides methods that correspond to certain of the uses set forth in the preceding paragraphs.

In certain aspects, the disclosure provides methods of modifying the flavor of an ingestible composition, comprising introducing any conjugated diynes of the preceding aspects or embodiments to an ingestible composition. In some embodiments, the ingestible composition is a food or beverage product.

In certain aspects, the disclosure provides methods of enhancing a salty taste of an ingestible composition, comprising introducing any conjugated diynes of the preceding aspects or embodiments to the ingestible composition. In a related aspect, the disclosure provides methods of reducing salt (e.g., sodium chloride) content of an ingestible composition, the method comprising introducing any conjugated diynes of the preceding aspects or embodiments to the ingestible composition. In some embodiments, the ingestible composition is a food or beverage product.

In certain aspects, the disclosure provides methods of enhancing an umami taste of an ingestible composition, comprising introducing any conjugated diynes of the preceding aspects or embodiments to the ingestible composition. In a related aspect, the disclosure provides methods of reducing or eliminating glutamate (e.g., monosodium glutamate) or aspartate content of an ingestible composition, the method comprising introducing any conjugated diynes of the preceding aspects or embodiments to the ingestible composition. In some embodiments, the ingestible composition is a food or beverage product.

In certain aspects, the disclosure provides methods of enhancing a kokumi taste of an ingestible composition, comprising introducing any conjugated diynes of the preceding aspects or embodiments to the ingestible composition. In a related aspect, the disclosure provides methods of reducing or eliminating glutamyl (e.g., L-glutamyl peptides) content of an ingestible composition, the method comprising introducing any compounds of the first or second aspects to the ingestible composition. In another related aspect, the disclosure provides methods of reducing or eliminating animal (e.g., animal broth or meat) content of an ingestible composition, the method comprising introducing any conjugated diynes of the preceding aspects or embodiments to the ingestible composition. In some embodiments, the ingestible composition is a food or beverage product.

In certain aspects, the disclosure provides methods of enhancing a warming or heating effect of an ingestible composition, comprising introducing any conjugated diynes of the preceding aspects or embodiments to the ingestible composition. In some embodiments, the ingestible composition is a food or beverage product.

In certain aspects, the disclosure provides methods of enhancing a cooling effect of an ingestible composition, comprising introducing any conjugated diynes of the preceding aspects or embodiments to the ingestible composition. In some embodiments, the ingestible composition is a food or beverage product. In some embodiments, the ingestible composition is an oral care product, such as a mouthwash, a toothpaste, a whitening agent, a dentifrice, and the like. In some embodiments, the ingestible composition comprises menthol.

In certain aspects, the disclosure provides methods of enhancing a sweet taste of an ingestible composition, comprising introducing any conjugated diynes of the preceding aspects or embodiments to the ingestible composition. In a related aspect, the disclosure provides methods of reducing or eliminating sweetener (e.g., sucrose, fructose, sucralose, etc.) content of an ingestible composition, the method comprising introducing any conjugated diynes of the preceding aspects or embodiments to the ingestible composition. In some embodiments, the ingestible composition is a food or beverage product.

In certain aspects, the disclosure provides methods of reducing a sour taste of an ingestible composition, comprising introducing any conjugated diynes of the preceding aspects or embodiments to the ingestible composition. In some embodiments, the ingestible composition is a food or beverage product.

In certain aspects, the disclosure provides methods of reducing a bitter taste of an ingestible composition, comprising introducing any conjugated diynes of the preceding aspects or embodiments to the ingestible composition. In some embodiments, the ingestible composition is a food or beverage product.

The foregoing uses and methods generally involve the use or introduction of the conjugated diynes to an ingestible composition having one or more additional components or ingredients. For example, in at least one aspect, the disclosure provides compositions comprising any conjugated diynes of the foregoing aspects.

In certain particular embodiments, the ingestible composition comprises monosodium glutamate and an conjugated diyne (or comestibly acceptable salts thereof). In some such embodiments, the introduction of the conjugated diyne (or comestibly acceptable salt thereof) permits the use of less monosodium glutamate (such as more than 10% less, more than 20% less, more than 30% less, more than 40% less, more than 50% less, more than 60% less, or more than 70% less, or more than 80% less, or more than 90% less) and still achieve a level of umami taste of a comparable product that employs a higher concentration of monosodium glutamate. In some related embodiments, the use of the conjugated diynes, or its comestibly acceptable salts, permits the elimination of monosodium glutamate from the composition. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises fat, such as animal or vegetable fat, and the conjugated diyne (or comestibly acceptable salts thereof). In some such embodiments, the introduction of the conjugated diyne (or comestibly acceptable salt thereof) permits one to use less fat (such as more than 10% less, more than 20% less, more than 30% less, more than 40% less, more than 50% less, more than 60% less, or more than 70% less, or more than 80% less, or more than 90% less) and still achieve a level of umami characteristic of a comparable product that employs a higher concentration of fat. In some related embodiments, the use of the conjugated diynes, or its comestibly acceptable salts, permits the elimination of fat from the composition. In some embodiments, the concentration of the conjugated diynes, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The fat can be any suitable fat, such as a fat derived from an animal or vegetable fat, including, but not limited to, milk fat (including fat in various cheeses), beef fat, pork fat, poultry fat, lamb fat, goat fat, fish oil, olive oil, canola oil, corn oil, safflower oil, nut oil, peanut oil, cashew oil, soybean oil, palm oil, palm kernel oil, coconut oil, butter, and nut butters (such as peanut butter, cashew butter, almond butter, hazelnut butter, and the like).

In certain particular embodiments, the ingestible composition comprises glutamate (including in its free acid form), and the conjugated diyne (or comestibly acceptable salts thereof). In some such embodiments, the introduction of the conjugated diyne (or comestibly acceptable salt thereof) permits one to use less glutamate (such as more than 10% less, more than 20% less, more than 30% less, more than 40% less, more than 50% less, more than 60% less, or more than 70% less, or more than 80% less, or more than 90% less) and still achieve a level of umami characteristic of a comparable product that employs a higher concentration of glutamate. In some related embodiments, the use of the conjugated diyne, or its comestibly acceptable salts, permits the elimination of glutamate from the composition. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The glutamate can be from any suitable source, such as monosodium glutamate, proteins containing glutamic acid (e.g., glutathione), and the like.

In certain particular embodiments, the ingestible composition comprises aspartate (including in its free acid form), and the conjugated diyne (or comestibly acceptable salts thereof). In some such embodiments, the introduction of the conjugated diyne (or comestibly acceptable salt thereof) permits one to use less aspartate (such as more than 10% less, more than 20% less, more than 30% less, more than 40% less, more than 50% less, more than 60% less, or more than 70% less, or more than 80% less, or more than 90% less) and still achieve a level of umami characteristic of a comparable product that employs a higher concentration of aspartate. In some related embodiments, the use of the conjugated diyne, or its comestibly acceptable salts, permits the elimination of aspartate from the composition. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The aspartate can be from any suitable source, such as proteins containing aspartic acid, and the like.

In certain particular embodiments, the ingestible composition comprises animal products, and the conjugated diyne (or comestibly acceptable salts thereof). In some such embodiments, the introduction of the conjugated diyne (or comestibly acceptable salt thereof) permits one to use less animal products (such as more than 10% less, more than 20% less, more than 30% less, more than 40% less, more than 50% less, more than 60% less, or more than 70% less, or more than 80% less, or more than 90% less) and still achieve a level of umami characteristic of a comparable product that employs a higher concentration of animal products. In some related embodiments, the use of the conjugated diyne, or its comestibly acceptable salts, permits the elimination of animal products from the composition. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The animal products can be any suitable animal product, such as cheese, milk, meat broth (such as beef broth, pork broth, chicken broth, turkey broth, duck broth, lamb broth, goat broth, rabbit broth, and the like), eggs, bone broth, bone marrow, meat (such as beef, pork, chicken, lamb, goat, turkey, duck, rabbit, and the like), butter, and animal skin.

In certain particular embodiments, the ingestible composition comprises vegetable products, and the conjugated diynes (or comestibly acceptable salts thereof). In some such embodiments, the introduction of the conjugated diyne (or comestibly acceptable salt thereof) permits one to use less vegetable product (such as more than 10% less, more than 20% less, more than 30% less, more than 40% less, more than 50% less, more than 60% less, or more than 70% less, or more than 80% less, or more than 90% less) and still achieve a level of umami characteristic of a comparable product that employs a higher concentration of vegetable products. In some related embodiments, the use of the conjugated diyne, or its comestibly acceptable salts, permits the elimination of vegetable products from the composition. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The vegetable products can be any suitable vegetable product, such as celery, celeriac, tomato, garlic, onion, leek, scallion, spices, and the like.

In certain particular embodiments, the ingestible composition comprises sodium (i.e., sodium cation), and the conjugated diyne (or comestibly acceptable salts thereof). In some such embodiments, the introduction of the conjugated diyne (or comestibly acceptable salt thereof) permits one to use less sodium (such as more than 10% less, more than 20% less, more than 30% less, more than 40% less, more than 50% less, more than 60% less, or more than 70% less, or more than 80% less, or more than 90% less) and still achieve a level of salty characteristic of a comparable product that employs a higher concentration of sodium. In some related embodiments, the use of the conjugated diyne, or its comestibly acceptable salts, permits the elimination of sodium from the composition. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The sodium can be any suitable animal product, such as table salt (sodium chloride), sea salt, soy sauce, fish sauce, shrimp paste, butter, miso, and Worcestershire sauce.

In certain particular embodiments, the ingestible composition comprises alcohol, and the conjugated diyne (or comestibly acceptable salts thereof). In some such embodiments, the introduction of the conjugated diyne (or comestibly acceptable salt thereof) permits one to use less alcohol (such as more than 10% less, more than 20% less, more than 30% less, more than 40% less, more than 50% less, more than 60% less, or more than 70% less, or more than 80% less, or more than 90% less) and still achieve a level of umami and/or kokumi characteristic of a comparable product that employs a higher concentration of alcohol. In some related embodiments, the use of the conjugated diyne, or its comestibly acceptable salts, permits the elimination of alcohol from the composition. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda (such as a hard soda), and the like. The alcohol can present in any suitable form, such as alcohol formed from grains, cane sugar, fruits, and the like.

In some instances, one may be able to reduce the amount of sweetener in a product by enhancing the umami or kokumi taste.

In certain particular embodiments, the ingestible composition comprises sucrose and the conjugated diyne or any of its comestibly acceptable salts. In some such embodiments, the introduction of the conjugated diyne (or salt) permits one to use less sucrose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more sucrose. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. In some embodiments, the ingestible composition is a food product, such as any of those specifically listed below. In other embodiments, the ingestible composition is a beverage product, such as a soda, and the like.

In certain particular embodiments, the ingestible composition comprises fructose and the conjugated diyne or any of its comestibly acceptable salts. In some such embodiments, the introduction of the conjugated diyne (or salt) permits one to use less fructose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more fructose. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. In some embodiments, the ingestible composition is a food product, such as any of those specifically listed below. In other embodiments, the ingestible composition is a beverage product, such as a soda, and the like.

In certain particular embodiments, the ingestible composition comprises high-fructose corn syrup and the conjugated diyne or any of its comestibly acceptable salts. In some such embodiments, the introduction of the conjugated diyne (or salt) permits one to use less high-fructose corn syrup (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more high-fructose corn syrup. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. In some embodiments, the ingestible composition is a food product, such as any of those specifically listed below. In other embodiments, the ingestible composition is a beverage product, such as a soda, and the like.

In certain particular embodiments, the ingestible composition comprises glucose (for example, D-glucose, in either its alpha or beta forms, or a combination thereof) and the conjugated diyne or any of its comestibly acceptable salts. In some such embodiments, the introduction of the conjugated diyne (or salt) permits one to use less glucose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more glucose. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The glucose can be introduced in any suitable form, such as natural syrups and the like.

In certain particular embodiments, the ingestible composition comprises sucralose and the conjugated diyne or any of its comestibly acceptable salts. In some such embodiments, the introduction of the conjugated diyne (or salt) permits one to use less sucralose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more sucralose. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises rebaudiosides (such as rebaudioside A, rebaudioside D, rebaudioside E, rebaudioside M, or any combination thereof) and the conjugated diyne or any of its comestibly acceptable salts. In some such embodiments, the introduction of the conjugated diyne (or salt) permits one to use less rebaudioside (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more rebaudioside. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises acefulfame K and the conjugated diyne or any of its comestibly acceptable salts. In some such embodiments, the introduction of the conjugated diyne (or salt) permits one to use less acesulfame K (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more acesulfame K. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises allulose and the conjugated diyne or any of its comestibly acceptable salts. In some such embodiments, the introduction of the conjugated diyne (or salt) permits one to use less allulose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more allulose. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises erythritol and the conjugated diyne or any of its comestibly acceptable salts. In some such embodiments, the introduction of the conjugated diyne (or salt) permits one to use less erythritol (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more erythritol. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises aspartame and the conjugated diyne or any of its comestibly acceptable salts. In some such embodiments, the introduction of the conjugated diyne (or salt) permits one to use less aspartame (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more aspartame. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises cyclamate and the conjugated diyne or any of its comestibly acceptable salts. In some such embodiments, the introduction of the conjugated diyne (or salt) permits one to use less cyclamate (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more cyclamate. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises a mogroside (such as mogroside III, mogroside IV, mogroside V, siamenoside I, isomogroside V, mogroside IV_{E}, isomogroside IV_{E}, isomogroside IV, mogroside III_{E}, 11-oxomogroside V, the 1,6-alpha isomer of siamenoside I, and any combinations thereof) and the conjugated diyne or any of its comestibly acceptable salts. In some such embodiments, the introduction of the conjugated diyne (or salt) permits one to use less a mogroside (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more mogroside. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. Additional mogroside compounds that may be suitably used are described in U.S. Patent Application Publication No. 2017/0119032.

In some other aspects, the disclosure provides use of the conjugated diyne, or a comestibly acceptable salt thereof, to enhance or confer an umami taste of an ingestible composition. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm, in the ingestible composition. Such ingestible compositions can be in any suitable form. In some embodiments, the ingestible composition is a food product, such as any of those specifically listed below. In other embodiments, the ingestible composition is a beverage product, such as a soda, and the like.

In some other aspects, the disclosure provides use of the conjugated diyne, or a comestibly acceptable salt thereof, to enhance or confer a kokumi taste of an ingestible composition. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm, in the ingestible composition. Such ingestible compositions can be in any suitable form. In some embodiments, the ingestible composition is a food product, such as any of those specifically listed below. In other embodiments, the ingestible composition is a beverage product, such as a soda, and the like.

In some other aspects, the disclosure provides use of the conjugated diyne, or a comestibly acceptable salt thereof, to enhance or confer a salty taste of an ingestible composition. In some embodiments, the concentration of the conjugated diyne, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm, in the ingestible composition. Such ingestible compositions can be in any suitable form. In some embodiments, the ingestible composition is a food product, such as any of those specifically listed below. In other embodiments, the ingestible composition is a beverage product, such as a soda, and the like.

In certain embodiments of any aspects and embodiments set forth herein that refer to an ingestible composition, the ingestible composition is a non-naturally-occurring product, such as a composition specifically manufactured for the production of a flavored product, such as food or beverage product.

In general, compounds as disclosed and described herein, individually or in combination, can be provided in a composition, such as an ingestible composition. In one embodiment, compounds as disclosed and described herein, individually or in combination, can impart a more sugar-like temporal profile or flavor profile to a sweetener composition by combining one or more of the compounds as disclosed and described herein with one or more sweeteners in the sweetener composition. In another embodiment, compounds as disclosed and described herein, individually or in combination, can increase or enhance the sweet taste of a composition by contacting the composition thereof with the compounds as disclosed and described herein to form a modified composition.

Thus, in some embodiments, the compositions set forth in any of the foregoing aspects (including in any uses or methods), comprise an conjugated diyne (of any aspects or embodiments set forth herein) and a sweetener. In some embodiments, the composition further comprises a vehicle. In some embodiments, the vehicle is water. In some embodiments, the conjugated diyne is present at a concentration at or below its umami or saltiness recognition threshold.

For example, in some embodiments, the sweetener (according to any of the embodiments set forth above) is present in an amount from about 0.1% to about 12% by weight. In some embodiments, the sweetener is present in an amount from about 0.2% to about 10% by weight. In some embodiments, the sweetener is present in an amount from about 0.3% to about 8% by weight. In some embodiments, the sweetener is present in an amount from about 0.4% to about 6% by weight. In some embodiments, the sweetener is present in an amount from about 0.5% to about 5% by weight. In some embodiments, the sweetener is present in an amount from about 1% to about 2% by weight. In some embodiments, the sweetener is present in an amount from about 0.1% to about 5% by weight. In some embodiments, the sweetener is present in an amount from about 0.1% to about 4% by weight. In some embodiments, the sweetener is present in an amount from about 0.1% to about 3% by weight. In some embodiments, the sweetener is present in an amount from about 0.1% to about 2% by weight. In some embodiments, the sweetener is present in an amount from about 0.1% to about 1% by weight. In some embodiments, the sweetener is present in an amount from about 0.1% to about 0.5% by weight. In some embodiments, the sweetener is present in an amount from about 0.5% to about 10% by weight. In some embodiments, the sweetener is present in an amount from about 2% to about 8% by weight. In some further embodiments of the embodiments set forth in this paragraph, the sweetener is sucrose, fructose, glucose, xylitol, erythritol, or combinations thereof.

In some other embodiments, the sweetener is present in an amount from 10 ppm to 1000 ppm. In some embodiments, the sweetener is present in an amount from 20 ppm to 800 ppm. In some embodiments, the sweetener is present in an amount from 30 ppm to 600 ppm. In some embodiments, the sweetener is present in an amount from 40 ppm to 500 ppm. In some embodiments, the sweetener is present in an amount from 50 ppm to 400 ppm. In some embodiments, the sweetener is present in an amount from 50 ppm to 300 ppm. In some embodiments, the sweetener is present in an amount from 50 ppm to 200 ppm. In some embodiments, the sweetener is present in an amount from 50 ppm to 150 ppm. In some further embodiments of the embodiments set forth in this paragraph, the sweetener is a steviol glycoside, a mogroside, a derivative of either of the foregoing, such as glycoside derivatives (e.g., glucosylates), or any combination thereof.

The compositions can include any suitable sweeteners or combination of sweeteners. In some embodiments, the sweetener is a common saccharide sweeteners, such as sucrose, fructose, glucose, and sweetener compositions comprising natural sugars, such as corn syrup (including high fructose corn syrup) or other syrups or sweetener concentrates derived from natural fruit and vegetable sources. In some embodiments, the sweetener is sucrose, fructose, or a combination thereof. In some embodiments, the sweetener is sucrose. In some other embodiments, the sweetener is selected from rare natural sugars including D-allose, D-psicose, L-ribose, D-tagatose, L-glucose, L-fucose, L-arbinose, D-turanose, and D-leucrose. In some embodiments, the sweetener is selected from semi-synthetic “sugar alcohol” sweeteners such as erythritol, isomalt, lactitol, mannitol, sorbitol, xylitol, maltodextrin, and the like. In some embodiments, the sweetener is selected from artificial sweeteners such as aspartame, saccharin, acesulfame-K, cyclamate, sucralose, and alitame. In some embodiments, the sweetener is selected from the group consisting of cyclamic acid, mogroside, tagatose, maltose, galactose, mannose, sucrose, fructose, lactose, allulose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A, other sweet Stevia-based glycosides, chemically modified steviol glycosides (such as glucosylated steviol glycosides), mogrosides, chemically modified mogrosides (such as glucosylated mogrosides), carrelame and other guanidine-based sweeteners. In some embodiments, the sweetener is a combination of two or more of the sweeteners set forth in this paragraph. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a sugar. In some embodiments, the sugar is cane sugar. In some embodiments, the sugar is beet sugar. In some embodiments, the sugar may be sucrose, fructose, glucose or combinations thereof. In some embodiments, the sugar may be sucrose. In some embodiments, the sugar may be a combination of fructose and glucose.

The sweetener can also include, for example, sweetener compositions comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi-synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

The sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, Stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

In still other embodiments, the sweetener can be a chemically or enzymatically modified natural high potency sweetener. Modified natural high potency sweeteners include glycosylated natural high potency sweetener such as glucosyl-, galactosyl-, or fructosyl-derivatives containing 1-50 glycosidic residues. Glycosylated natural high potency sweeteners may be prepared by enzymatic transglycosylation reaction catalyzed by various enzymes possessing transglycosylating activity. In some embodiments, the modified sweetener can be substituted or unsubstituted.

Additional sweeteners also include combinations of any two or more of any of the aforementioned sweeteners. In some embodiments, the sweetener may comprise combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a caloric sweetener, such as sucrose, fructose, xylitol, erythritol, or combinations thereof. In some embodiments, the ingestible compositions are free (or, in some embodiments) substantially free of stevia-derived sweeteners, such as steviol glycosides, glucosylated steviol glycosides, or rebaudiosides. For example, in some embodiments, the ingestible compositions are either free of stevia-derived sweeteners or comprise stevia-derived sweeteners in a concentration of no more than 1000 ppm, or no more than 500 ppm, or no more than 200 ppm, or no more than 100 ppm, or no more than 50 ppm, or no more than 20 ppm, or no more than 10 ppm, or no more than 5 ppm, or no more than 3 ppm, or no more than 1 ppm.

The conjugated diyne can be present in the ingestible compositions in any suitable amount. In some embodiments, the conjugated diyne are present in an amount sufficient to enhance the taste (e.g., enhance the umami, enhance the kokumi, enhance the saltiness, reduce the sourness, or reduce the bitterness) of the compositions. Thus, in some embodiments, the ingestible composition comprises the conjugated diynes in a concentration no greater than 200 ppm, or no greater than 150 ppm, or no greater than 100 ppm, or no greater than 50 ppm, or no greater than 40 ppm, or no greater than 30 ppm, or no greater than 20 ppm. In some embodiments, the conjugated diyne is present in a minimum amount, such as 1 ppm or 5 ppm. Thus, in some embodiments, the ingestible composition comprises the conjugated diynes in a concentration ranging from 1 ppm to 200 ppm, or from 1 ppm to 150 ppm, or from 1 ppm to 100 ppm, or from 1 ppm to 50 ppm, or from 1 ppm to 40 ppm, or from 1 ppm to 30 ppm, or from 1 ppm to 20 ppm, or from 5 ppm to 200 ppm, or from 5 ppm to 150 ppm, or from 5 ppm to 100 ppm, or from 5 ppm to 50 ppm, or from 5 ppm to 40 ppm, or from 5 ppm to 30 ppm, or from 5 ppm to 20 ppm. In embodiments where a sweetener, such as sucrose or fructose, are present, the weight-to-weight ratio of sweetener to the conjugated diyne in the ingestible composition ranges from 1000:1 to 50000:1, or from 1000:1 to 10000:1, or from 2000:1 to 8000:1.

The ingestible compositions or sweetener concentrates can, in certain embodiments, comprise any additional ingredients or combination of ingredients as are commonly used in food and beverage products, including, but not limited to:
acids, including, for example citric acid, phosphoric acid, ascorbic acid, sodium acid sulfate, lactic acid, or tartaric acid;
bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);
coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;
preservatives, including, for example sodium benzoate, potassium benzoate, potassium sorbate, sodium metabisulfate, sorbic acid, or benzoic acid;
antioxidants including, for example ascorbic acid, calcium disodium EDTA, alpha tocopherols, mixed tocopherols, rosemary extract, grape seed extract, resveratrol, or sodium hexametaphosphate;
vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, Ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;
clouding agents, including, for example ester gun, brominated vegetable oil (BVO), or sucrose acetate isobutyrate (SAIB);
buffers, including, for example sodium citrate, potassium citrate, or salt;
flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; or
starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

The ingestible compositions or sweetener concentrates can have any suitable pH. In some embodiments, the conjugated diynes enhance the sweetness of a sweetener under a broad range of pH, e.g., from lower pH to neutral pH. The lower and neutral pH includes, but is not limited to, a pH from 1.5 to 9.0, or from 2.5 to 8.5; from 3.0 to 8.0; from 3.5 to 7.5; and from 4.0 to 7. In certain embodiments, compounds as disclosed and described herein, individually or in combination, can enhance the perceived sweetness of a fixed concentration of a sweetener in taste tests at a compound concentration of 50 μM, 40 μM, 30 μM, 20 μM, or 10 μM at both low to neutral pH value. In certain embodiments, the enhancement factor of the compounds as disclosed and described herein, individually or in combination, at the lower pH is substantially similar to the enhancement factor of the compounds at neutral pH. Such consistent sweet enhancing property under a broad range of pH allow a broad use in a wide variety of foods and beverages of the compounds as disclosed and described herein, individually or in combination.

The ingestible compositions set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that reduce sourness or licorice taste, compounds that enhance saltiness, compounds that enhance a cooling effect, or any combinations of the foregoing.

Thus, in some embodiments, ingestible compositions disclosed herein comprise the conjugated diyne, or any comestibly acceptable salts thereof, according to any of the embodiments or combination of embodiments set forth above, are combined with one or more sweetness enhancing compounds. Such sweetness enhancing compounds include, but are not limited to, naturally derived compounds, such as hesperitin, naringenin, rhoifolin, glucosylated steviol glycosides, licorice-derived glucuronates, aromadendrin-3-O-acetate, or other like flavonols, or flavonoids, or synthetic compounds, such as any compounds set forth in U.S. Pat. Nos. 8,541,421; 8,815,956; 9,834,544; 8,592,592; 8,877,922; 9,000,054; and 9,000,051, as well as U.S. Patent Application Publication No. 2017/0119032. The conjugated diynes (or comestibly acceptable salts thereof) may be used in combination with such other sweetness enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1. In some embodiments of any of the preceding embodiments, the conjugated diyne (or any comestibly acceptable salts thereof) is combined with glucosylated steviol glycosides in any of the above ratios. As used herein, the term “glucosylated steviol glycoside” refers to the product of enzymatically glucosylating natural steviol glycoside compounds. The glucosylation generally occurs through a glycosidic bond, such as an α-1,2 bond, an α-1,4 bond, an α-1,6 bond, a β-1,2 bond, a μ-1,4 bond, a β-1,6 bond, and so forth. In some embodiments of any of the preceding embodiments, the conjugated diyne (or any comestibly acceptable salts thereof) is combined with 3-((4-amino-2,2-dioxo-1H-benzo[c][1,2,6]thiadiazin-5-yl)oxy)-2,2-dimethyl-N-propyl-propanamide, N-(1-((4-amino-2,2-dioxo-1H-benzo[c][1,2,6]thiadiazin-5-yl)oxy)-2-methyl-propan-2-yl)isonicotinamide, or any combination thereof, in any of the above ratios.

In some further embodiments, ingestible compositions disclosed herein comprise the conjugated diyne, or any comestibly acceptable salts thereof, according to any of the embodiments or combination of embodiments set forth above, are combined with one or more other umami or kokumi enhancing compounds. Such umami enhancing compounds include, but are not limited to, naturally derived compounds, such as ericamide, or synthetic compounds, such as any compounds set forth in U.S. Pat. Nos. 8,735,081; 8,124,121; and 8,968,708. The conjugated diyne (or comestibly acceptable salts thereof) may be used in combination with such umami enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the conjugated diyne, or any comestibly acceptable salts thereof, according to any of the embodiments or combination of embodiments set forth above, are combined with one or more cooling enhancing compounds. Such cooling enhancing compounds include, but are not limited to, naturally derived compounds, such as menthol or analogs thereof, or synthetic compounds, such as any compounds set forth in U.S. Pat. Nos. 9,394,287 and 10,421,727. The conjugated diyne (or comestibly acceptable salts thereof) may be used in combination with such umami enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the conjugated diyne, or any comestibly acceptable salts thereof, according to any of the embodiments or combination of embodiments set forth above, are combined with one or more bitterness blocking compounds. Such bitterness blocking compounds include, but are not limited to, naturally derived compounds, such as menthol or analogs thereof, or synthetic compounds, such as any compounds set forth in U.S. Pat. Nos. 8,076,491; 8,445,692; and 9,247,759. The conjugated diyne (or comestibly acceptable salts thereof) may be used in combination with such bitterness blockers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the conjugated diyne, or any comestibly acceptable salts thereof, according to any of the embodiments or combination of embodiments set forth above, are combined with one or more sour taste modulating compounds. The conjugated diyne (or comestibly acceptable salts thereof) may be used in combination with such sour taste modulating compounds in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the conjugated diyne, or any comestibly acceptable salts thereof, according to any of the embodiments or combination of embodiments set forth above, are combined with one or more mouthfeel modifying compounds. Such mouthfeel modifying compounds include, but are not limited to, tannins, cellulosic materials, bamboo powder, and the like. The conjugated diyne (or comestibly acceptable salts thereof) may be used in combination with such mouthfeel enhancers in any suitable ratio (w/w) ranging from 1:1000 to 1000:1, or from 1:100 to 100:1, or fro, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the conjugated diyne, or any comestibly acceptable salts thereof, according to any of the embodiments or combination of embodiments set forth above, are combined with one or more flavor masking compounds. Such flavor masking compounds include, but are not limited to, cellulosic materials, materials extracted from fungus, materials extracted from plants, citric acid, carbonic acid (or carbonates), and the like. The conjugated diyne (or comestibly acceptable salts thereof) may be used in combination with such mouthfeel enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some aspects related to the preceding aspects and embodiments, the disclosure provides uses of the conjugated diyne (or comestibly acceptable salts thereof) to enhance the flavor of a flavored composition, such as a flavored article. Such flavored compositions can use any suitable flavors, such as fruit flavors, meat flavors, vegetable flavors, and the like. In some embodiments, the flavored composition is a soup or broth, or a chip, or a beverage.

### Flavored Products and Concentrates

In certain aspects, the disclosure provides flavored products comprising any compositions of the preceding aspects or embodiments thereof. In some embodiments, the flavored products are beverage products, such as soda, flavored water, tea, and the like. In some other embodiments, the flavored products are food products, such as yogurt.

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

In certain embodiments of any aspects and embodiments set forth herein that refer to a flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommd) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal's milk. Examples of dairy product include, but are not limited to drinking milk products, cheese, yoghurt and sour milk drinks, and other dairy products.

Additional examples for flavored products, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including compounds as disclosed and described herein, individually or in combination.

Typically at least a sweet receptor modulating amount, a sweet receptor ligand modulating amount, a sweet flavor modulating amount, a sweet flavoring agent amount, a sweet flavor enhancing amount, or a therapeutically effective amount of one or more of the present compounds will be added to the ingestible composition, optionally in the presence of sweeteners so that the sweet flavor modified ingestible composition has an increased sweet taste as compared to the ingestible composition prepared without the compounds of the present invention, as judged by human beings or animals in general, or in the case of formulations testing, as judged by a majority of a panel of at least eight human taste testers, via procedures commonly known in the field.

In some embodiments, compounds as disclosed and described herein, individually or in combination, modulate the sweet taste or other taste properties of other natural or synthetic sweet tastants, and ingestible compositions made therefrom. In one embodiment, the compounds as disclosed and described herein, individually or in combination, may be used or provided in its ligand enhancing concentration(s). For example, the compounds as disclosed and described herein, individually or in combination, may be present in an amount of from 0.001 ppm to 100 ppm, or narrower alternative ranges from 0.1 ppm to 50 ppm, from 0.01 ppm to 40 ppm, from 0.05 ppm to 30 ppm, from 0.01 ppm to 25 ppm, or from 0.1 ppm to 30 ppm, or from 0.1 ppm to 25 ppm, or from 1 ppm to 30 ppm, or from 1 ppm to 25 ppm.

In some embodiments, conjugated diynes as disclosed and described herein, individually or in combination, may be provided in a flavoring concentrate formulation, e.g., suitable for subsequent processing to produce a ready-to-use (i.e., ready-to-serve) product. By “a flavoring concentrate formulation”, it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

The flavored products set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that reduce sourness, compounds that enhance saltiness, compounds that enhance a cooling effect, or any combinations of the foregoing.

In certain embodiments of any aspects and embodiments set forth herein that refer to a sweetening or flavoring concentrate, the sweetening or flavoring concentrate is a non-naturally-occurring product, such as a composition specifically manufactured for the production of a flavored product, such as food or beverage product.

In one embodiment, the flavoring concentrate formulation comprises i) compounds as disclosed and described herein, individually or in combination; ii) a carrier; and iii) optionally at least one adjuvant. The term “carrier” denotes a usually inactive accessory substance, such as solvents, binders, or other inert medium, which is used in combination with the present compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described in this application. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

In one embodiment, the flavoring concentrate formulation has the present compound in a concentration that is at least 2 times of the concentration of the compound in a ready-to-use composition. In one embodiment, the flavoring concentrate formulation has the present compound in a concentration that is at least 5 times of the concentration of the compound in a ready-to-use composition. In one embodiment, the flavoring concentrate formulation has the present compound in a concentration that is at least 10 times of the concentration of the compound in a ready-to-use composition. In one embodiment, the flavoring concentrate formulation has the present compound in a concentration that is at least 15 times of the concentration of the compound in a ready-to-use composition. In one embodiment, the flavoring concentrate formulation has the present compound in a concentration that is at least 20 times of the concentration of the compound in a ready-to-use composition. In one embodiment, the flavoring concentrate formulation has the present compound in a concentration that is at least 30 times of the concentration of the compound in a ready-to-use composition. In one embodiment, the flavoring concentrate formulation has the present compound in a concentration that is at least 40 times of the concentration of the compound in a ready-to-use composition. In one embodiment, the flavoring concentrate formulation has the present compound in a concentration that is at least 50 times of the concentration of the compound in a ready-to-use composition. In one embodiment, the flavoring concentrate formulation has the present compound in a concentration that is at least 60 times of the concentration of the compound in a ready-to-use composition. In one embodiment, the flavoring concentrate formulation has the present compound in a concentration that is up to 100 times of the concentration of the compound in a ready-to-use composition.

The sweetening or flavoring concentrates set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness (e.g., eriodictyol, homoeriodictyol, sterubin, and salts or glycoside derivatives thereof, as well as vanillyl lignans, e.g., matairesinol and other compounds set forth in PCT Publication No. WO 2012/146584), compounds that enhance umami (e.g., rubemamine, rubescenamine, (E)-3-(3,4-dimethoxyphenyl)-N-(4-methoxyphenethyl)acrylamide, and the like), compounds that reduce sourness and/or licorice taste, compounds that enhance saltiness, compounds that enhance a cooling effect, or any combinations of the foregoing.

### Tabletop Flavoring Compositions

In some further aspects, the disclosure provides a tabletop flavoring composition comprising: (a) an conjugated diyne (according to any aspects and embodiments set forth herein), or a comestibly acceptable salt thereof; and (b) at least one bulking agent.

The tabletop flavoring composition may take any suitable form including, but not limited to, an amorphous solid, a crystal, a powder, a tablet, a liquid, a cube, a glace or coating, a granulated product, an encapsulated form abound to or coated on to carriers/particles, wet or dried, or combinations thereof.

The tabletop flavoring composition may contain further additives known to those skilled in the art. These additives include but are not limited to bubble forming agents, bulking agents, carriers, fibers, sugar alcohols, oligosaccharides, sugars, high intensity sweeteners, nutritive sweeteners, flavorings, flavor enhancers, flavor stabilizers, acidulants, anti-caking and free-flow agents. Such additives are for example described by H. Mitchell (H. Mitchell, “Sweeteners and Sugar Alternatives in Food Technology”, Blackwell Publishing Ltd, 2006, which is incorporated herein by reference in its entirety). As used herein, the term “flavorings” may include those flavors known to the skilled person, such as natural and artificial flavors. These flavorings may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya and so forth. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with cooling agents.

Flavors may also provide breath freshening properties, particularly the mint flavors when used in combination with cooling agents. These flavorings may be used in liquid or solid form and may be used individually or in admixture. Other useful flavorings include aldehydes and esters such as cinnamyl acetate, cinnamaldehyde, citral diethylacetal, dihydrocarvyl acetate, eugenyl formate, p-methylamisol, and so forth may be used. Generally any flavoring or food additive such as those described in Chemicals Used in Food Processing, publication 1274, pages 63-258, by the National Academy of Sciences, may be used. This publication is incorporated herein by reference.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), cherry, grape, strawberry shortcake, and mixtures thereof. These listings of flavorings are merely exemplary and are not meant to limit either the term “flavoring” or the scope of the disclosure generally.

In some embodiments, the flavoring may be employed in either liquid form and/or dried form. When employed in the latter form, suitable drying means such as spray drying the oil may be used. Alternatively, the flavoring may be absorbed onto water soluble materials, such as cellulose, starch, sugar, maltodextrin, gum arabic and so forth or may be encapsulated. The actual techniques for preparing such dried forms are well-known.

In some embodiments, the tabletop sweetener can be made to be similar to brown sugar. In such embodiments, compounds imparting brown notes can be added to the composition to make it taste more similar to brown sugar.

In some embodiments, the flavorings may be used in many distinct physical forms well-known in the art to provide an initial burst of flavor and/or a prolonged sensation of flavor. Without being limited thereto, such physical forms include free forms, such as spray dried, powdered, beaded forms, encapsulated forms, and mixtures thereof.

Suitable bulking agents include, but are not limited to maltodextrin (10 DE, 18 DE, or 5 DE), corn syrup solids (20 or 36 DE), sucrose, fructose, glucose, invert sugar, sorbitol, xylose, ribulose, mannose, xylitol, mannitol, galactitol, erythritol, maltitol, lactitol, isomalt, maltose, tagatose, lactose, inulin, glycerol, propylene glycol, polyols, polydextrose, fructooligosaccharides, cellulose and cellulose derivatives, and the like, and mixtures thereof. Additionally, granulated sugar (sucrose) or other caloric sweeteners such as crystalline fructose, other carbohydrates, or sugar alcohols can be used as a bulking agent due to their provision of good content uniformity without the addition of significant calories.

In one embodiment, the at least one bulking agent may be a bulking agent described in U.S. Pat. No. 8,993,027.

In one embodiment, the at least one bulking agent may be a bulking agent described in U.S. Pat. No. 6,607,771.

In one embodiment, the at least one bulking agent may be a bulking agent described in U.S. Pat. No. 6,932,982.

In some embodiments, the tabletop sweetener composition may further comprise at least one anti-caking agent. As used herein the phrase “anti-caking agent” and “flow agent” refer to any composition which prevents, reduces, inhibits, or suppresses the at least one sweetener from attaching, binding, or contacting to another sweetener molecule. Alternatively, anti-caking agent may refer to any composition which assists in content uniformity and uniform dissolution. Non-limiting examples of anti-caking agents include cream of tartar, calcium silicate, silicon dioxide, microcrystalline cellulose (Avicel, FMC BioPolymer, Philadelphia, Pa.), and tricalcium phosphate. In one embodiment, the anti-caking agents are present in the tabletop sweetener composition in an amount from about 0.001 to about 3% by weight of the tabletop sweetener composition.

In some embodiments, the sweetener compositions of any of the preceding aspects and embodiments thereof are encapsulated using typical means for encapsulating flavor or fragrance compounds. Non-limiting examples of such technology are set forth in U.S. Patent Application Publication Nos. 2016/0235102, 2019/0082727, 2018/0369777, 2018/0103667, 2016/0346752, 2015/0164117, 2014/0056836, 2012/0027866, 2010/0172945, and 2007/0128234, as well as U.S. Pat. Nos. 7,488,503, 6,416,799, 5,897,897, 5,786,017, 5,603,971, 4,689,235, 4,610,890, 3,704,137, 3,041,180, and 2,809,895. All of the preceding patent publications and patents are hereby incorporated by reference as though set forth herein in their entireties.

### Non-Animal Protein Materials and Products Made Therefrom

Products intended to replace or substitute meat or dairy products often rely on various non-animal-based materials, such as starches and proteins derived from plants, algae, and fungi, to simulate the texture and flavor of meat or dairy. Non-limiting examples of such plant proteins include soy proteins, pea proteins, bean proteins, grain proteins, and the like. Due to compositional differences between such plant-based materials and animal-derived materials, such as a lack of glutamate-containing proteins and glutathione, these products can lack the umami and/or kokumi taste that consumers traditionally associate with meat or dairy products.

Thus, in certain aspects, the disclosure provides a flavored product comprising a plant-based material (such as a plant-based starch, a plant-based protein, or a combination thereof) and an conjugated diyne (according to any aspects and embodiments set forth herein), or a comestibly acceptable salt thereof. In some further embodiments, the flavored product can include any features of combination of features set forth above for ingestible compositions that contain the conjugated diyne, or a comestibly acceptable salt thereof. In some embodiments, the flavored product is a beverage, such as soy milk, almond milk, rice milk, oat milk, a protein drink, a meal-replacement drink, or other like product. In some other embodiments, the flavored product is a meat-replacement product, such as a plant-based chicken product (such as a plant-based chicken nugget), a plant-based beef product (such as a plant-based burger), and the like. In some other embodiments, the flavored product is a protein powder, a meal-replacement powder, a plant-based creamer for coffee or tea, and the like. In certain further embodiments, any such products contain additional ingredients, and have additional features, as are typically used in the preparation and/or manufacture of such products. For example, such an conjugated diyne, or comestibly acceptable salts thereof, may be combined with other flavors and taste modifiers, and may even be encapsulated in certain materials, according to known technologies in the relevant art. Suitable concentrations of the conjugated diyne, or comestibly acceptable salts thereof, are set forth above.

In some further embodiments analogous to the above embodiments, proteins or starches from algal or fungal sources can be used instead of or in combination with plant starches or proteins.

### Non-Meat Protein Materials and Products Made Therefrom

Certain non-meat animal proteins, such as dairy proteins and proteins from bone broth, are commonly used in food products, and are also sold as the primary ingredient in certain protein powders. Such proteins can impart flavors that lack the full umami or kokumi taste that consumers may desire. This is especially true for protein isolates, such as protein isolates of whey protein, collagen protein, casein proteins, and the like. Thus, the present disclosure provides ingestible compositions that include non-meat animal proteins and the conjugated diyne (according to any aspects and embodiments set forth herein), or a comestibly acceptable salt thereof. The conjugated diyne, or its comestibly acceptable salts, can be present in any suitable combination, according to the embodiments set forth in the preceding sections of the present disclosure. In some embodiments, the non-meat animal protein is a bone protein, such as a collagen protein derived from the bones of an animal, such as a cow, pig, donkey, horse, chicken, duck, goat, goose, rabbit, lamb, sheep, buffalo, ostrich, camel, and the like. In some embodiments, the non-meat animal protein is a milk protein, such as a whey protein, a casein protein, or any combination thereof. The milk can be the milk of any suitable animal, such as a cow, donkey, horse, sheep, buffalo, camel, and the like.

The conjugated diyne, or its comestibly acceptable salts, can also be included in certain food or beverage products that include animal milk or materials derived from animal milk. Such products include cheeses, cheese spreads, yogurt, kefir, milk, processed dairy products, cottage cheese, sour cream, butter, and the like.

### Methods of Preparation

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary or desirable to protect sensitive or reactive groups on any of the molecules concerned.

### EXAMPLES

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1—Product Isolation

5.5 Kg of fresh stems and leaves of Chrysanthemum nankingense were cut into pieces and extracted with 95% ethanol. The solution was concentrated by a rotor vaporator under reduced pressure and loaded on an MCI column. The column was rinsed with water, 60% and 90% ethanol to get three fractions, Fr1, Fr2, and Fr3. Fr3 was further subjected to silica gel column elution by cyclohexane and ethyl acetate in gradient to afford four sub-fractions. The third sub-fraction, which contained most of the alkylamides, was chosen for further separation with preparative LC chromatography. Solvent systems A (water) and B (acetonitrile) were used for the prep-HPLC separation on a phenomenex C18(2) column (150*21.20 mm*5 μm). Gradient elution program (60% B to 70% B) was used for each separation. Peaks were collected using a fraction collector at UV 250 nm. Three alkylamides were obtained: nona-6,8-diynoic acid piperidide (Compound 101, 505 mg), nona-6,8-diynoic acid piperideide (Compound 102, 60 mg) and (E,E)-2,4-undecadien-8,10-diynoic acid piperidide (Compound 130, 485 mg). The structures were determined by NMR and HR ESI MS spectrum. The molecular weight of the [M+H]+ molecular ion was determined as follows for the three compounds:
Compound 101 218.1537 amu;
Compound 102 216.1378 amu; and
Compound 130 242.1537 amu.
   Table 2 below provides the ¹H and ¹³C NMR spectrum data for Compound 101, Compound 102, and Compound 130.

TABLE 2

| | |
|---|---|
| Compound 101 | ¹H-NMR (600 MHz, CDCl3; δ (ppm): 1.50-1.59 (m; 4H, C(3′)HH and |
| ¹H NMR | C(5′)HH); 1.57-1.63 (m; 2H, C(4)HH); 1.60-1.67 (m; 2H, C(4′)HH); |
| | 1.71-1.77 (m; 2H, C(3)HH); 1.97 (t, 1H, J = 1.2 Hz; C(9)H); 2.31 (dt, |
| | 2H, J = 7.0, 1.2 Hz; C(5)HH); 2.34 (t, 2H, J = 7.5 Hz; C(2)HH); 3.39 (m, |
| | 2H; C(2′)HH and C(6′)HH); 3.54 (m, 2H; C(2′)HH and C(6′)HH). |
| Compound 101 | ¹³C-NMR (150 MHz, CDCl3); δ (ppm): 18.9 (t, CH2(5)); 24.5 (t, |
| ¹³C NMR | CH2(3)); 24.6 (t, CH2(4′)); 25.6 and 26.6 (t, CH2(3′) and CH2(5′)); |
| | 27.7 (t, CH2(4)); 32.7 (t, CH2(2)); 42.7 (t, CH2(2′)); 46.7 (t, CH2(6′)); |
| | 64.7 (d, CH (9)); 65.0 (s, C (7)); 68.4 (s, C (8)); 78.0 (s, C (6)); 170.8 (s, |
| | C═O). |
| Compound 102 | ¹H-NMR (600 MHz, CDCl3; δ (ppm): 1.57-1.64 (m; 2H, C(4)HH); |
| ¹H NMR | 1.74-1.80 (m; 2H, C(3)HH); 1.79-1.84 (m; 2H, C(5′)HH); 1.97 (t, 1H, J = |
| | 1.2 Hz, C(9)H); 2.06-2.10 (m; 2H, C(4′)HH); 2.31 (dt, 2H, J = 6.9, 1.4 |
| | Hz; C(5)HH); 2.34 (t, 2H, J = 7.5 Hz; C(2)HH); 3.66-3.70 (m; 2H, |
| | C(6′)HH); 4.97 and 5.08 (1H, dt, J = 4.0, 8.2 Hz; C(3′)H); 6.60 and 6.73 |
| | (1H, dt, J = 1.8, 8.3 Hz; br d, J = 8.5 Hz; C(2′)H). |
| Compound 102 | ¹³C-NMR (150 MHz, CDCl3); δ (ppm): 18.9 (t, CH2(5)); 21.6 (t, |
| ¹³C NMR | CH2(5′)); 21.9 (t, CH2(4′)); 24.0 (t, CH2(3)); 27.6 (t, CH2(4)); 32.6 (t, |
| | CH2(2)); 40.3 (t, CH2(6′)); 64.9 (d, CH (9)); 65.1 (s, C (7)); 68.4 (s, C |
| | (8)); 77.9 (s, C(6)); 108.3 (d, CH (3′)); 125.2 (d, CH (2′)); 170.0 (s, |
| | C═O). |
| Compound 130 | ¹H-NMR (600 MHz, CDCl3; δ (ppm): 1.55-1.59 (m; 4H, C(3′)HH and |
| ¹H NMR | C(5′)HH); 1.63-1.68 (m, 2H, C(4′)HH) 2.00 (t, 1H, J = 1.0 Hz, |
| | C(11)H); 2.38-2.40 (m, 4H, C(6)HH and C(7)HH); 3.49 (br s, 2H; |
| | C(2′)HH and C(6′)HH); 3.61 (br s, 2H; C(2′)HH and C(6′)HH); 6.02 |
| | (dt, 1H, J = 15.1, 6.9 Hz; C(5)H); 6.24 (dd, 1H, J = 10.9, 15.1 Hz; |
| | C(4)H); 6.31 (d, 1H, J = 14.7 Hz; C(2)H); 7.18 (dd, 1H, J = 10.9, 14.7 |
| | Hz; C(3)H). |
| Compound 130 | ¹³C-NMR (150 MHz, CDCl3); δ (ppm): 18.8 (t, CH2(7)); 24.6 (t, |
| ¹³C NMR | CH2(4′)); 25.6 and 26.7 (t, CH2(3′) and CH2(5′)); 31.3 (t, CH2(6)); |
| | 43.2 (t, CH2(2′)); 46.9 (t, CH2(6′)); 65.1 (d, CH (11)); 65.4 (s, C (9)); |
| | 68.3 (s, C (10)); 77.0 (s, C (8)); 120.0 (d, CH (2)); 130.4 (d, CH (4)); |
| | 138.4 (d, CH (5)); 141.9 (d, CH (3)); 165.4 (s, C═O). |

### Example 2—Synthesis of Compounds 103-118

N-bromosuccinimide (NBS, 10.3 g), silver nitrate (8.56 g), and triisopropylsilane-acetylene (TIPS-acetylene) (11.2 mL) were added to 400 mL of acetone (pre-dried). The solution was stirred vigorously for 3.5 hours at room temperature. Once the reaction reached completion (as shown by GC-MS), 300 mL of water was added. The resulting solution was extracted into cyclohexane (3×500 mL). The combined organic layer was washed with brine (2×500 mL), dried over anhydrous magnesium sulfate, filtered, concentrated, and dried further via a rotary evaporator to give a clear oil product (13.4247 g), which was determined to be 1-(bromoethynyl)triisopropylsilane (Product 2a).

Copper (I) chloride (156 mg) was added to a 30% n-BuNH₂ (butylamine) aqueous solution (130 mL) at room temperature, which resulted in the formation of a blue solution. A few crystals of hydroxylamine hydrochloride were added to discharge the blue color. After putting the solution in an ice-water bath, 6-heptynoic acid (3.7 g) in ethyl ether (50 mL) was added dropwise to the cooled reaction mixture. Occasionally adding a small amount of hydroxylamine hydrochloride was necessary to keep the color of the reaction a light yellow. After adding Product 2a (usually 30 minutes), the cooling bath was removed. The reaction mixture was stirred at room temperature for another 2 hours during which more crystals of hydroxylamine hydrochloride was added whenever the reaction mixture started to turn blue or green. Once the reaction completed (as shown by GC-MS), the reaction mixture was quenched and adjusted to a pH of about 2 by adding 2N hydrochloric acid (250 mL). The resulting solution was extracted into ethyl acetate (3×400 mL). The combined organic layer was washed with brine (1×500 mL), dried over magnesium sulfate, filtered, concentrated, and dried via a rotary evaporator to give a product of 9-TIPS-6,8-diynoic acid (Product 2b).

Under an atmosphere of nitrogen gas, tetra-n-butylammonium fluoride (TBAF) (1M solution in tetrahydrofuran (THF), 45 mL) was added dropwise to a solution of Product 2b (9.2 g) in 66 mL of THF at 0° C. The solution was stirred at room temperature for 3-4 hours. Once the reaction reached completion (as shown by GC-MS), the reaction mixture was quenched with 2N Hydrochloric acid and adjusted to a pH of about 2 (150 mL). The reaction product was extracted with dichloromethane (DCM) (3×500 mL). The combined organic layers were washed with brine (2×500 mL), dried over magnesium sulfate, filtered, concentrated and dried via rotary evaporator to give a crude product. The total crude product was purified by flash chromatography (CHA/DCM) to afford nona-6,8-diynoic acid (3.9201 g) (Product 2c).

The resulting amides Compounds 103-118 were formed by amidating Product 2c with the appropriate amine. The amines used were as follows: Compound 103 (isobutylamine); Compound 104 (2-methylbutylamine); Compound 105 (beta-phenethylamine); Compound 106 (4-hydroxy-3-methoxybenzylamine); Compound 107 (4-hydroxy-4-methoxybenzylamine); Compound 108 (benzylamine); Compound 109 (butylamine); Compound 110 (tyramine); Compound 111 (4-methoxyphenethylamine); Compound 112 (ethanolamine); Compound 113 (4-hydroxybenzylamine); Compound 114 (3,4-dimethoxyphenethylamine); Compound 115 (2-hydroxy-2-methylpropylamine); Compound 116 (3,4-methylenedioxy-phenethylamine); Compound 117 (1-aminopropan-2-one); and Compound 118 (pyrrolidine).

The molecular weight of the [M+H]+ molecular ion was determined as follows for the following compounds:
Compound 103 206.1547 amu;
Compound 104 220.1705 amu;
Compound 105 254.1547 amu;
Compound 106 286.1446 amu;
Compound 107 286.1447 amu;
Compound 108 240.1393 amu;
Compound 109 206.1546 amu;
Compound 110 270.1495 amu;
Compound 111 284.1657 amu;
Compound 112 194.1181 amu;
Compound 113 256.1338 amu;
Compound 114 314.1759 amu;
Compound 115 222.1486 amu;
Compound 116 298.1433 amu;
Compound 117 206.1173 amu; and
Compound 118 204.1380 amu.
   Table 3 below provides the ¹H and ¹³C NMR spectrum data for Compounds 103-118.

TABLE 3

| | |
|---|---|
| Compound 103 | ¹H-NMR (600 MHz, CDCl3; δ (ppm): 0.91 and 0.92 (2d, 6H, J = 6.6 |
| ¹H NMR | Hz, CH(2′)CH₃CH₃); 1.56-1.62 (m; 2H, C(4)HH); 1.73-1.78 (m; 2H, |
| | C(3)HH); 1.74-1.79 (m; 2H, C(2′)HH); 1.97 (s, 1H, C(9)H); 2.20 (t, 2H, |
| | J = 7.5 Hz; C(2)HH); 2.30 (dt, 2H, J = 6.9, 1.2 Hz; C(5)HH); 3.09 (t, 2H, |
| | J = 6.4; C(1′)HH). |
| Compound 103 | ¹³C-NMR (150 MHz, CDCl₃); δ (ppm): 18.9 (t, CH₂(5)); 20.1 (2q, |
| ¹³C NMR | CH₃(3′) and CH₃(4′)); 24.9 (t, CH₂(3)); 27.5 (t, CH₂(4)); 28.5 (d, CH |
| | (2′)); 36.2 (t, CH₂(2)); 46.9 (t, CH₂(1′)); 64.8 (d, CH (9)); 65.1 (s, C |
| | (7)); 68.4 (s, C (8)); 77.9 (s, C (6)); 172.4 (s, C═O). |
| Compound 104 | ¹H-NMR (600 MHz, CDCl3; δ (ppm): 0.89 (d; 3H, J = 6.7 Hz; CHCH₃); |
| ¹H NMR | 0.90 (t; 3H, J = 7.4 Hz; CH₂CH₃); 1.11-1.19 (m; H, CHHCH₃); 1.35-1.43 |
| | (m; H, CHHCH₃); 1.54-1.57 (m; H, CHCH₃); 1.56-1.61 (m; 2H, |
| | C(4)HH); 1.72-1.78 (m; 2H, C(3)HH); 1.97 (s, 1H, C(9)H); 2.20 (t, 2H, |
| | J = 7.3 Hz; C(2)HH); 2.29 (dt, 2H, J = 6.9, 1.4 Hz; C(5)HH); 3.07 (1H, |
| | ddd, J = 13.4, 6.1, 7.1 Hz; C(1′)HH); 3.20 (1H, ddd, J = 13.4, 6.1, 6.1 |
| | Hz; C(1′)HH). |
| Compound 104 | ¹³C-NMR (150 MHz, CDCl3;); δ (ppm): 11.3 ((q, CH₃(4′)); 17.2 ((q, |
| ¹³C NMR | CH₃(5′)); 18.9 (t, CH₂(5)); 24.9 (t, CH₂(3)); 27.0 (t, CH₂(3′)); 27.5 (t, |
| | CH₂(4)); 34.9 (d, CH(2′)); 36.2 (t, CH₂(2)); 45.1 (t, CH₂(1′)); 64.7 (d, |
| | CH (9)); 65.1 (s, C (7)); 68.4 (s, C (8)); 77.9 (s, C (6)); 172.4 (s, C═O). |
| Compound 105 | ¹H-NMR (600 MHz, CDCl3; δ (ppm): 1.51-1.56 (m; 2H, C(4)HH); |
| ¹H NMR | 1.67-1.73 (m; 2H, C(3)HH); 1.97 (s, 1H, C(9)H); 2.14 (t, 2H, J = 7.4 Hz; |
| | C(2)HH); 2.26 (t, 2H, J = 6.9 Hz; C(5)HH); 2.81 (t, 2H, J = 6.9 Hz; |
| | C(7′)HH); 3.52 (dt, 2H, J = 6.9, 6.5 Hz; C(8′)HH); 7.18-7.20 (m; 2H, |
| | C(2′)H and C(6′)H); 7.22-7.26 (m; H, C(4′)H); 7.30-7.33 (m; 2H, |
| | C(2′)H and C(6′)H). |
| Compound 105 | ¹³C-NMR (150 MHz, CDCl3;); δ (ppm): 18.8 (t, CH₂(5)); 24.8 (t, |
| ¹³C NMR | CH₂(3)); 27.4 (t, CH₂(4)); 35.7 (t, CH₂(7′)); 36.0 (t, CH₂(2)); 40.5 (t, |
| | CH₂(8′)); 64.8 (d, CH (9)); 65.1 (s, C (7)); 68.4 (s, C (8)); 77.8 (s, C |
| | (6)); 126.5 (d, CH (4′)); 128.7 (2d, CH (3′) and CH (5′)); 128.8 (2d, |
| | CH (2′) and CH (6′)); 138.8 (s, C (1′)); 172.4 (s, C═O). |
| Compound 106 | ¹H-NMR (600 MHz, CDCl3; δ (ppm): 1.56-1.61 (m; 2H, C(4)HH); |
| ¹H NMR | 1.74-1.80 (m; 2H, C(3)HH); 1.97 (s, 1H, C(9)H); 2.22 (t, 2H, J = 7.5 Hz; |
| | C(2)HH); 2.29 (t, 2H, J = 6.9 Hz; C(5)HH); 3.88 (s; 3H, OCH3); 4.34- |
| | 4.36 (m; 2H, C(7′)HH); 6.76 (1H, dd, J = 7.7, 2.1 Hz; C(6′)H); 6.80 |
| | (1H, d, J = 2.1 Hz; C(2′)H); 6.86 (1H, d, J = 7.7 Hz; C(5′)H). |
| Compound 106 | ¹³C-NMR (150 MHz, Methanol-d4;); δ (ppm): 18.9 (t, CH₂(5)); 24.9 (t, |
| ¹³C NMR | CH₂(3)); 27.5 (t, CH₂(4)); 36.1 (t, CH₂(2)); 43.8 (t, CH₂(7′)); 56.0 (q, |
| | OCH₃); 64.8 (d, CH (9)); 65.1 (s, C (7)); 68.3 (s, C (8)); 77.8 (s, C (6)); |
| | 110.7 (d, CH (2′)); 114.4 (d, CH (5′)); 120.8 (d, CH (6′)); 130.2 (s, C |
| | (1′)); 145.2 (d, CH (3′)); 146.7 (d, CH (4′)); 172.2 (s, C═O). |
| Compound 107 | ¹H-NMR (600 MHz, Methanol-d4; δ (ppm): 1.51-1.57(m; 2H, C(4)HH); |
| ¹H NMR | 1.69-1.74 (m; 2H, C(3)HH); 2.23 (t, 2H, J = 7.3 Hz; C(2)HH); 2.29 (dt, |
| | 2H, J = 6.9, 1.2 Hz; C(5)HH); 2.5 (s, 1H, C(9)H); 3.82 (s; 3H, OCH₃); |
| | 4.22 (s; 2H, C(7′)HH); 6.71 (1H, dd, J = 8.0, 2.0 Hz; C(6′)H); 6.74 (1H, |
| | d, J = 2.0 Hz; C(2′)H); 6.85 (1H, d, J = 8.0 Hz; C(5′)H). |
| Compound 107 | ¹³C-NMR (150 MHz, CDCl3); δ (ppm): 19.3 (t, CH₂(5)); 26.2 (t, |
| ¹³C NMR | CH₂(3)); 28.8 (t, CH₂(4)); 36.5 (t, CH₂(2)); 43.8 (t, CH₂(7′)); 56.5 (q, |
| | OCH₃); 66.0 (s, C (7)); 66.5 (d, CH (9)); 69.1 (s, C (8)); 78.1 (s, C (6)); |
| | 112.8 (d, CH (5′)); 115.9 (d, CH (2′)); 120.0 (d, CH (6′)); 132.9 (s, C |
| | (1′)); 147.7 (d, CH (3′)); 148.3 (d, CH (4′)); 175.5 (s, C═O). |
| Compound 108 | ¹H-NMR (600 MHz, CDCl3; δ (ppm): 1.56-1.62 (m; 2H, C(4)HH); |
| ¹H NMR | 1.75-1.81 (m; 2H, C(3)HH); 1.97 (s, 1H, C(9)H); 2.24 (t, 2H, J = 7.4 Hz; |
| | C(2)HH); 2.30 (t, 2H, J = 6.9 Hz; C(5)HH); 4.43-4.45 (m; 2H, |
| | C(7′)HH); 7.25-7.29 (m; 2H, C(2′)H and C(6′)H); 7.27-7.30 (m; H, |
| | C(4′)H); 7.32-7.35 (m; 2H, C(2′)H and C(6′)H). |
| Compound 108 | ¹³C-NMR (150 MHz, CDCl3;); δ (ppm): 18.9 (t, CH₂(5)); 24.8 (t, |
| ¹³C NMR | CH₂(3)); 27.5 (t, CH₂(4)); 36.0 (t, CH₂(2)); 43.7 (t, CH₂(7′)); 64.8 (d, |
| | CH (9)); 65.1 (s, C (7)); 68.4 (s, C (8)); 77.8 (s, C (6)); 127.6 (d, CH |
| | (4′)); 127.9 (2d, CH (2′) and CH (6′)); 128.8 (2d, CH (3′) and CH (5′)); |
| | 138.3 (s, C (1′)); 172.2 (s, C═O). |
| Compound 109 | ¹H-NMR (600 MHz, CDCl3; δ (ppm): 0.93 (t; 3H, J = 7.4 Hz; CH₂CH₃); |
| ¹H NMR | 1.31-1.38 (m; 2H, CHHCH3); 1.45-1.51 (m; 2H, CHHCH₂CH₃); 1.55- |
| | 1.61 (m; 2H, C(4)HH); 1.72-1.78 (m; 2H, C(3)HH); 1.97 (1H, C(9)H); |
| | 2.18 (t, 2H, J = 7.5 Hz; C(2)HH); 2.29 (t, 2H, J = 6.9,; C(5)HH); ); 3.25 |
| | (dt, 2H, J = 7.2, 6.9,; C(1′)HH). |
| Compound 109 | ¹³C-NMR (150 MHz, CDCl3;); δ (ppm): 13.8 (q, CH₃(4′)); 18.9 (t, |
| ¹³C NMR | CH₂(5)); 20.1 (t, CH₂(3′)); 24.9 (t, CH₂(3)); 27.5 (t, CH₂(4)); 31.7 (t, |
| | CH₂(2′)); 36.1 (t, CH₂(2)); 39.3 (t, CH₂(1′)); 64.7 (d, CH (9)); 65.1 (s, C |
| | (7)); 68.4 (s, C (8)); 77.9 (s, C (6)); 172.3 (s, C═O). |
| Compound 110 | ¹H-NMR (600 MHz, CDCl3; δ (ppm): 1.49-1.54 (m; 2H, C(4)HH); |
| ¹H NMR | 1.67-1.73 (m; 2H, C(3)HH); 1.98 (s, 1H, C(9)H); 2.15 (t, 2H, J = 7.6 Hz; |
| | C(2)HH); 2.26 (t, 2H, J = 7.1 Hz; C(5)HH); 2.74 (t, 2H, J = 7.1 Hz; |
| | C(7′)HH); 3.49 (dt, 2H, J = 7.1, 6.5 Hz; C(8′)HH); 6.79 (d; 2H, J = 8.4 |
| | Hz; C(3′)H and C(5′)H); 7.03 (d; 2H, J = 8.4 Hz; C(2′)H and C(6′)H). |
| Compound 110 | ¹³C-NMR (150 MHz, CDCl3;); δ (ppm): 18.8 (t, CH₂(5)); 24.8 (t, |
| ¹³C NMR | CH₂(3)); 27.4 (t, CH₂(4)); 34.7 (t, CH₂(7′)); 36.1 (t, CH₂(2)); 40.8 (t, |
| | CH₂(8′)); 64.8 (d, CH(9)); 65.1 (s, C(7)); 68.4 (s, C(8)); 77.9 (s, C (6)); |
| | 115.6 (2d, CH (3′) and CH (5′)); 129.8 (2d, CH(2′) and CH (6′)); 130.2 |
| | (s, C(1′)); 154.8 (s, C(4′)); 172.8 (s, C═O). |
| Compound 111 | ¹H-NMR (600 MHz, CDCl3; δ (ppm): 1.51-1.56 (m; 2H, C(4)HH); |
| ¹H NMR | 1.68-1.74 (m; 2H, C(3)HH); 1.97 (s, 1H, C(9)H); 2.14 (t, 2H, J = 7.6 Hz; |
| | C(2)HH); 2.27 (t, 2H, J = 7.0 Hz; C(5)HH); 2.75 (t, 2H, J = 6.9 Hz; |
| | C(7′)HH); 3.48 (dt, 2H, J = 6.9, 6.4 Hz; C(8′)HH); 6.85 (d; 2H, J = 8.4 |
| | Hz; C(3′)H and C(5′)H); 7.10 (d; 2H, J = 8.4 Hz; C(2′)H and C(6′)H). |
| Compound 111 | ¹³C-NMR (150 MHz, CDCl3;); δ (ppm): 18.9 (t, CH₂(5)); 24.8 (t, |
| ¹³C NMR | CH₂(3)); 27.5 (t, CH₂(4)); 34.8 (t, CH₂(7′)); 36.0 (t, CH₂(2)); 40.7 (t, |
| | CH₂(8′)); 55.3 (q, OCH₃); 64.8 (d, CH (9)); 65.1 (s, C (7)); 68.4 (s, C |
| | (8)); 77.8 (s, C (6)); 114.1 (2d, CH (3′) and CH (5′)); 129.7 (2d, CH |
| | (2′) and CH (6′)); 130.8 (s, C (1′)); 158.3 (s, C (4′)); 172.3 (s, C═O). |
| Compound 112 | ¹H-NMR (600 MHz, methanol-d4; δ (ppm): 1.52-1.57 (m; 2H, C(4)HH); |
| ¹H NMR | 1.68-1.73 (m; 2H, C(3)HH); 2.51 (s, 1H, C(9)H); 2.22 (t, 2H, J = 7.5 Hz; |
| | C(2)HH); 2.30 (t, 2H, J = 6.8; C(5)HH); 3.28 (t, 2H, J = 5.8; C(1′)HH); |
| | 3.58 (t, 2H, J = 5.8; C(2′)HH). |
| Compound 112 | ¹³C-NMR (150 MHz, methanol-d4;); δ (ppm): 19.3 (t, CH₂(5)); 26.1 (t, |
| ¹³C NMR | CH₂(3)); 28.8 (t, CH₂(4)); 43.0 (t, CH₂(1′)); 36.4 (t, CH₂(2)); (d, CH (9), |
| | n.v.); 61.6(t, CH₂(2′)); 66.0 (s, C (7)); 68.8 (s, C (8)); 78.0 (s, C (6)); |
| | 176.1 (s, C═O). |
| Compound 113 | ¹H-NMR (600 MHz, CDCl3; δ (ppm): 1.55-1.61 (m; 2H, C(4)HH); |
| ¹H NMR | 1.74-1.80 (m; 2H, C(3)HH); 1.96 (s, 1H, C(9)H); 2.23 (t, 2H, J = 7.3 Hz; |
| | C(2)HH); 2.28 (dt, 2H, J = 6.9, 1.1 Hz; C(5)HH); 4.35 (d, 2H, J = 5.5 |
| | Hz; C(7′)HH); 6.79 (d; 2H, J = 8.4 Hz; C(3′)H and C(5′)H); 7.13 (d; |
| | 2H, J = 8.4 Hz; C(2′)H and C(6′)H). |
| Compound 113 | ¹³C-NMR (150 MHz, CDCl3;); δ (ppm): 18.9 (t, CH₂(5)); 24.8 (t, |
| ¹³C NMR | CH₂(3)); 27.5 (t, CH₂(4)); 36.0 (t, CH₂(2)); 43.2 (t, CH₂(7′)); 64.8 (d, |
| | CH (9)); 65.1 (s, C (7)); 68.4 (s, C (8)); 77.8 (s, C (6)); 115.6 (2d, CH |
| | (3′) and CH (5′)); 129.4 (2d, CH (2′) and CH (6′)); 130.1 (s, C (1′)); |
| | 155.4 (s, C (4′)); 172.4 (s, C═O). |
| Compound 114 | ¹H-NMR (600 MHz, CDCl₃; δ (ppm): 1.52-1.58(m; 2H, C(4)HH); 1.69- |
| ¹H NMR | 1.74 (m; 2H, C(3)HH); 1.97 (s, 1H, C(9)H); 2.15 (t, 2H, J = 7.4 Hz; |
| | C(2)HH); 2.28 (dt, 2H, J = 6.9, 1.1 Hz; C(5)HH); 3.87 and 3.88 (2s; 6H, |
| | C(3′)OCH₃ and C(4′)OCH₃); 2.76 (t, 2H, J = 6.9 Hz; C(7′)HH); 3.50 |
| | (dt, 2H, J = 6.9, 6.5 Hz; C(8′)HH); 6.73 (1H, dd, J = 8.0, 2.0 Hz; |
| | C(6′)H); 6.72 (1H, d, J = 2.0 Hz; C(2′)H); 6.82 (1H, d, J = 8.0 Hz; |
| | C(5′)H). |
| Compound 114 | ¹³C-NMR (150 MHz, CDCl3;); δ (ppm): 18.9 (t, CH₂(5)); 24.8 (t, |
| ¹³C NMR | CH₂(3)); 27.5 (t, CH₂(4)); 35.3 (t, CH₂(7′)); 36.1 (t, CH₂(2)); 40.6 (t, |
| | CH₂(8′)); 56.0 (q, C(3′)OCH₃); 55.9 (q, C(4′)OCH₃); 64.8 (d, CH (9)); |
| | 65.1 (s, C (7)); 68.4 (s, C (8)); 77.8 (s, C (6)); 111.4 (d, CH (5′)); 111.9 |
| | (d, CH (2′)); 120.7 (d, CH (6′)); 131.3 (s, C (1′)); 147.7 (s, C (4′)); |
| | 149.1 (s, C (3′)); 172.3 (s, C═O). |
| Compound 115 | ¹H-NMR (600 MHz, CDCl₃; δ (ppm): 1.22 (2s; 6H, OC CH₃CH₃); 1.57- |
| ¹H NMR | 1.62(m; 2H, C(4)HH); 1.74-1.80 (m; 2H, C(3)HH); 1.99 (s, 1H, C(9)H); |
| | 2.26 (t, 2H, J = 7.5 Hz; C(2)HH); 2.30 (t, 2H, J = 6.9 Hz; C(5)HH); |
| | 3.27(d, 2H, J = 5.8 Hz; C(3′)HH). |
| Compound 115 | ¹³C-NMR (150 MHz, CDCl3;); δ (ppm): 18.8 (t, CH₂(5)); 24.9 (t, |
| ¹³C NMR | CH₂(3)); 27.3 and 27.3 (2q, CH₃ and CH₃) 27.5 (t, CH₂(4)); 36.0 (t, |
| | CH₂(2)); 50.3 (t, CH₂(2′)); 64.8 (d, CH (9)); 65.1 (s, C (7)); 68.4 (s, C |
| | (8)); 70.9 (s, C (1′)); 77.8 (s, C (6)); 172.3 (s, C═O). |
| Compound 116 | ¹H-NMR (600 MHz, CDCl₃; δ (ppm): 1.52-1.57(m; 2H, C(4)HH); 1.69- |
| ¹H NMR | 1.74 (m; 2H, C(3)HH); 1.98 (s, 1H, C(9)H); 2.14 (t, 2H, J = 7.4 Hz; |
| | C(2)HH); 2.27 (dt, 2H, J = 6.9, 1.0 Hz; C(5)HH); 2.73 (t, 2H, J = 6.9 |
| | Hz; C(7′)HH); 3.46 (dt, 2H, J = 6.8, 6.5 Hz; C(8′)HH); 5.93 (2H, s, |
| | OCHHO)6.63 (1H, dd, J = 7.9, 1.4 Hz; C(2′)H); 6.68 (1H, d, J = 1.4 Hz; |
| | C(2′)H); 6.74 (1H, d, J = 7.9 Hz; C(6′)H). |
| Compound 116 | ¹³C-NMR (150 MHz, CDCl3;); δ (ppm): 18.8 (t, CH₂(5)); 24.8 (t, |
| ¹³C NMR | CH₂(3)); 27.4 (t, CH₂(4)); 35.4 (t, CH₂(7′)); 36.0 (t, CH₂(2)); 40.7 (t, |
| | CH₂(8′)); 64.8 (d, CH (9)); 65.1 (s, C (7)); 68.4 (s, C (8)); 77.8 (s, C |
| | (6)); 100.9 (t, OCH₂O) 109.0 (d, CH (2′)); 108.4 (d, CH (5′)); 121.6 (d, |
| | CH (6′)); 132.6 (s, C (1′)); 146.2 (s, C (4′)); 147.9 (s, C (3′)); 172.4 (s, |
| | C═O). |
| Compound 117 | ¹H-NMR (600 MHz, CDCl₃; δ (ppm): 1.56-1.62(m; 2H, C(4)HH); 1.73- |
| ¹H NMR | 1.79 (m; 2H, C(3)HH); 1.98 (s, 1H, C(9)H); 2.21 (s; 3H, O═CCH₃); 2.27 |
| | (t, 2H, J = 7.5 Hz; C(2)HH); 2.30 (dt, 2H, J = 6.9, 1.0 Hz; C(5)HH); |
| | 4.16 (d, 2H, J = 4.6 Hz; C(3′)HH). |
| Compound 117 | ¹³C-NMR (150 MHz, CDCl3;); δ (ppm): 18.8 (t, CH₂(5)); 24.7 (t, |
| ¹³C NMR | CH₂(3)); 27.4 (q, CH₃); 27.5 (t, CH₂(4)); 49.8 (t, CH₂(3′)); 64.8 (d, CH |
| | (9)); 65.1 (s, C (7)); 68.4 (s, C (8)); 77.8 (s, C (6)); 172.6 (s, C═O); |
| | 203.1 (s, C═O). |
| Compound 118 | ¹H-NMR (600 MHz, CDCl3; δ (ppm): 1.58-1.64 (m; 2H, C(4)HH); |
| ¹H NMR | 1.74-1.79 (m; 2H, C(3)HH); 1.86 (4H, tt, J = 6.9, 6.8 Hz, C(3′)HH); |
| | 1.96 (4H, tt, J = 6.8, 6.7 Hz, C(4′)HH); 1.97 (s, 1H,; C(9)H); 2.28 (t, |
| | 2H, J = 7.5,; C(2)HH); 2.30 (dt, 2H, J = 7.0, 1.0 Hz; C(5)HH); 3.41 (2H; |
| | t, J = 6.7 Hz C(5′)HH) and 3.46 (2H; t, J = 6.9 Hz C(2′)HH). |
| Compound 118 | ¹³C-NMR (150 MHz, CDCl3;); δ (ppm): 18.9 (t, CH₂(5)); 24.4 (t, |
| ¹³C NMR | CH₂(3′)); 24.0 (t, CH₂(3)); 26.1 (t, CH₂(4′)); 27.7 (t, CH₂(4)); 34.1 (t, |
| | CH₂(2)); 45.7 (t, CH₂(2′)); 46.6 (t, CH₂(5′)); 64.6 (d, CH (9)); 65.0 (s, C |
| | (7)); 68.4 (s, C (8)); 78.1 (s, C (6)); 171.1 (s, C═O). |

### Example 3—Sensory Testing for Salt Enhancement

Compounds 101 and 130 were evaluated for their salt-enhancement effects by a panel of 20 taste testers. The test compounds were added to solutions (0.25 wt %) sodium chloride, and tested for their sale enhancement effect relative to the same salt solution without the enhancer. Table 4 summarizes the results.

TABLE 4

| Cmpd | Conc. (ppm) | Difference | Remarks |
|---|---|---|---|
| 101 | 10 | None | No effect |
| 101 | 20 | + | Weak |
| 130 | 10 | ++ | Moderate |
| 130 | 20 | ++ | Moderate |
| 101 + | 20 | +++ | Strong |
| 130 | 20 | | |

### Example 4—Sensory Testing for Cooling/Burning

During tastings, 42 mg/L of Compound 130 in water solution was described as having a long-lasting warm, hot, weak cooling after taste, as well as bitter and astringent. At 20 mg/L, the taste attributes were selected as astringent, bitter, salty, earthy, herbal, and cooling, but no warm and hot sensation was perceived. At 2 mg/L, Compound 130 was tasteless.

A sample of 125 mg/L of Compound 101 in water solution was perceived as tingling (mid intensity), bitter, metallic, astringent and triggered an uncomfortable feeling in the throat when swallowed. At 39.2 mg/L, the tingling effect was weak. At 20 mg/L, the taste attributes of Compound 101 were perceived as astringent, bitter, herbal, earthy and green. At 2 mg/L, Compound 101 was tasteless.

1. A taste-modifying compound, where the compound is a compound of formula (I): 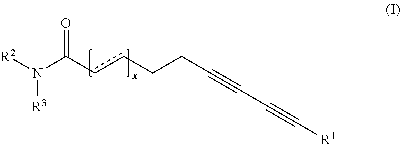
or a comestibly acceptable salt thereof;
wherein:
R¹ is a hydrogen atom, C₁₋₆ alkyl, C₂₋₆ alkenyl, C₃₋₁₀ cycloalkyl, C₂₋₁₀ heterocyclyl, C₆₋₁₄ aryl, C₁₋₁₂ heteroaryl, or —X¹—R⁴, wherein the alkyl and alkenyl groups are each optionally substituted one or more times by substituents selected independently from R^{X}, and wherein the cycloalkyl, heterocyclyl, aryl, and heteroaryl groups are each optionally substituted one or more times by substituents selected independently from the group consisting of R^{Y};
R² and R³ combine to form a heterocyclic or heteroaromatic ring comprising at least one nitrogen atom and from 2 to 10 carbon atoms, and wherein the ring is optionally substituted one or more times by substituents selected independently from R^{Y};
X¹ and X² are independently C₁₋₆ alkylene or C₂₋₆ alkenylene, each of which is optionally substituted one or more times by substituents selected independently from R^{X};
R⁴ and R⁵ are independently C₃₋₁₀ cycloalkyl, C₂₋₁₀ heterocyclyl, C₆₋₁₄ aryl, or C₁₋₁₂ heteroaryl, each of which is optionally substituted one or more times by substituents selected independently from R^{Y};
R^{X} is a halogen atom, oxo, —CN, nitro, —OH, —NH₂, —C(O)H, —O—C(O)H, —C(O)—OH, —NH—C(O)H, —C(O)—NH₂, —O—(C₁₋₆ alkyl), —NH—(C₁₋₆ alkyl), —N(C₁₋₆ alkyl)₂, —C(O)—(C₁₋₆ alkyl), —O—C(O)—(C₁₋₆ alkyl), —NH—C(O)—(C₁₋₆ alkyl), —C(O)—O—(C₁₋₆ alkyl), —C(O)—NH—(C₁₋₆ alkyl), —C(O)—N(C₁₋₆ alkyl)₂, —S(O)₂—(C₁₋₆ alkyl), —O—S(O)₂—(C₁₋₆ alkyl), —NH—S(O)₂—(C₁₋₆ alkyl), —S(O)₂—O—(C₁₋₆ alkyl), —S(O)₂—NH—(C₁₋₆ alkyl), —S(O)₂—N(C₁₋₆ alkyl)₂, C₃₋₁₀ cycloalkyl, C₂₋₁₄ heterocyclyl, C₆₋₁₄ aryl, C₂₋₁₄ heteroaryl, C₁₋₆ alkyl, C₂₋₆ alkenyl, C₁₋₆ haloalkyl, C₂₋₆ haloalkenyl, C₁₋₆ haloalkoxy, C₂₋₆ haloalkenyloxy, or (C₁₋₆ alkoxy)-C₁₋₆ alkyl;
R^{Y} is a halogen atom, oxo, —CN, nitro, —OH, —NH₂, —C(O)H, —O—C(O)H, —C(O)—OH, —NH—C(O)H, —C(O)—NH₂, —O—(C₁₋₆ alkyl), —NH—(C₁₋₆ alkyl), —N(C₁₋₆ alkyl)₂, —C(O)—(C₁₋₆ alkyl), —O—C(O)—(C₁₋₆ alkyl), —NH—C(O)—(C₁₋₆ alkyl), —C(O)—O—(C₁₋₆ alkyl), —C(O)—NH—(C₁₋₆ alkyl), —C(O)—N(C₁₋₆ alkyl)₂, —S(O)₂—(C₁₋₆ alkyl), —O—S(O)₂—(C₁₋₆ alkyl), —NH—S(O)₂—(C₁₋₆ alkyl), —S(O)₂—O—(C₁₋₆ alkyl), —S(O)₂—NH—(C₁₋₆ alkyl), —S(O)₂—N(C₁₋₆ alkyl)₂, C₃₋₁₀ cycloalkyl, C₂₋₁₄ heterocyclyl, C₆₋₁₄ aryl, C₂₋₁₄ heteroaryl, C₁₋₆ alkyl, C₂₋₆ alkenyl, C₁₋₆ haloalkyl, C₂₋₆ haloalkenyl, C₁₋₆ haloalkoxy, C₂₋₆ haloalkenyloxy, or (C₁₋₆ alkoxy)-C₁₋₆ alkyl; and
x is 1 or 2;
wherein the dotted-line bond indicates an optional carbon-carbon double bond, which can exist in either the E or Z configuration.

2. (canceled)

3. The compound of claim 1, wherein R¹ is a hydrogen atom.

4. (canceled)

5. The compound of claim 1, wherein x is 1, and the optional carbon-carbon double bond indicated by the dotted line is absent.

6. The compound of claim 1, wherein x is 2, and the optional carbon-carbon double bond indicated by the dotted line is present.

7. (canceled)

8. (canceled)

9. (canceled)

10. (canceled)

11. (canceled)

12. A method of modifying a flavor of an ingestible composition, the method comprising introducing a compound of claim 1 to the ingestible composition.

13. The method of claim 12, wherein modifying a flavor of an ingestible composition comprises enhancing a salty taste of an ingestible composition.

14. An ingestible composition, which comprises a compound of claim 1.

15. The ingestible composition of claim 14, further comprising sodium chloride.
